SEQUENCE LISTING (40 sequences)

SEQ ID NO: 1 - DNA, 41 bp, Artificial Sequence
  primer
aattatgagactgagaccacctctgttatcccagcagctcg

SEQ ID NO: 2 - DNA, 41 bp, Artificial Sequence
  primer
ttaatactctgactctggtggagacaatagggtcgtcgagc

SEQ ID NO: 3 - DNA, 23 bp, Artificial Sequence
  primer
tgagaccccctctgttatcccag

SEQ ID NO: 4 - DNA, 23 bp, Artificial Sequence
  primer
atactctgactctgggggagaca

SEQ ID NO: 5 - DNA, 15 bp, Artificial Sequence
  primer
gttgccaacgatcag

SEQ ID NO: 6 - DNA, 23 bp, Artificial Sequence
  primer
tgagaccccctctgttatcccag

SEQ ID NO: 7 - DNA, 23 bp, Artificial Sequence
  primer
acagagggggtctcagtctcata

SEQ ID NO: 8 - DNA, 31 bp, Artificial Sequence
  primer
gataccctctttccacaggttgcaccccaag

SEQ ID NO: 9 - DNA, 31 bp, Artificial Sequence
  primer
acctgtggaaagagggtatcgccatcaagga

SEQ ID NO: 10 - DNA, 23 bp, Artificial Sequence
  primer
aacatttcaggaaatggagggcc

SEQ ID NO: 11 - DNA, 23 bp, Artificial Sequence
  primer
tttcctgaaatgttttcaacact

SEQ ID NO: 12 - DNA, 23 bp, Artificial Sequence
  primer
ttaagaacatcagctttcccgaa

SEQ ID NO: 13 - DNA, 23 bp, Artificial Sequence
  primer
agctgatgttcttaatggttcca

SEQ ID NO: 14 - DNA, 23 bp, Artificial Sequence
  primer
ggaccatgcaaacttcaaataca

SEQ ID NO: 15 - DNA, 23 bp, Artificial Sequence
  primer
agtttgcatggtccacctcatca

SEQ ID NO: 16 - DNA, 23 bp, Artificial Sequence
  primer
tttccctcaggcctccctttcaa

SEQ ID NO: 17 - DNA, 23 bp, Artificial Sequence
  primer
aggcctgagggaaagctgatctt

SEQ ID NO: 18 - DNA, 24 bp, Artificial Sequence
  primer
tgaaggatctggagggcctggaac

SEQ ID NO: 19 - DNA, 24 bp, Artificial Sequence
  primer
ccctccagatccttcaatgttttc

SEQ ID NO: 20 - DNA, 24 bp, Artificial Sequence
  primer
ggcaactggtgatggaggatccat

SEQ ID NO: 21 - DNA, 24 bp, Artificial Sequence
  primer
ccatcaccagttgccactatcttt

SEQ ID NO: 22 - DNA, 15 bp, Artificial Sequence
  primer
catgccatccgtaag

SEQ ID NO: 23 - DNA, 41 bp, Artificial Sequence
  primer
accacagcctccagcgaagaatatgaaaaatttggtatgga

SEQ ID NO: 24 - DNA, 41 bp, Artificial Sequence
  primer
tggtgtcggaggtcgcttcttatactttttaaaccatacct

SEQ ID NO: 25 - DNA, 21 bp, Artificial Sequence
  primer
ccagcggctaatatgaaaaat

SEQ ID NO: 26 - DNA, 21 bp, Artificial Sequence
  primer
gtcggaggtcgccgattatac

SEQ ID NO: 27 - DNA, 41 bp, Artificial Sequence
  primer
ggctaatcaatgtcaatatggtcacgatacttgcagggatg

SEQ ID NO: 28 - DNA, 41 bp, Artificial Sequence
  primer
ccgattagttacagttataccagtgctatgaacgtccctac

SEQ ID NO: 29 - DNA, 21 bp, Artificial Sequence
  primer
tgtcaagctggtcacgatact

SEQ ID NO: 30 - DNA, 21 bp, Artificial Sequence
  primer
ttagttacagttcgaccagtg

SEQ ID NO: 31 - DNA, 21 bp, Artificial Sequence
  primer
ccagcggctaatatgaaaaat

SEQ ID NO: 32 - DNA, 21 bp, Artificial Sequence
  primer
catattagccgctggaggctg

SEQ ID NO: 33 - DNA, 21 bp, Artificial Sequence
  primer
tgtcaagctggtcacgatact

SEQ ID NO: 34 - DNA, 21 bp, Artificial Sequence
  primer
gtgaccagcttgacattgatt

SEQ ID NO: 35 - DNA, 21 bp, Artificial Sequence
  primer
attcatcagctgcgagatagg

SEQ ID NO: 36 - DNA, 480 bp, betula verrucosa
ggtgtgtttaattatgagactgagaccacctctgttatcccagcagctcgactgttcaaggcctttatccttgatggcgataacctctttccaaaggttgcaccccaagccattagcagtgttgaaaacattgaaggaaatggagggcctggaaccattaagaagatcagctttcccgaaggcctccctttcaagtacgtgaaggacagagttgatgaggtggaccacacaaacttcaaatacaattacagcgtgatcgagggcggtcccataggcgacacattggagaagatctccaacgagataaagatagtggcaacccctgatggaggatccatcttgaagatcagcaacaagtaccacaccaaaggtgaccatgaggtgaaggcagagcaggttaaggcaagtaaagaaatgggcgagacacttttgagggccgttgagagctacctcttggcacactccgatgcctacaactaa

SEQ ID NO: 37 - protein, 159 aa, betula verrucosa
GVFNYETETTSVIPAARLFKAFILDGDNLFPKVAPQAISSVENIEGNGGPGTIKKISFPEGLPFKYVKDRVDEVDHTNFKYNYSVIEGGPIGDTLEKISNEIKIVATPDGGSILKISNKYHTKGDHEVKAEQVKASKEMGETLLRAVESYLLAHSDAYN

SEQ ID NO: 38 - DNA, 615 bp, vespula vulgaris
aacaattattgtaaaataaaatgtttgaaaggaggtgtccatactgcctgcaaatatggaagtcttaaaccgaattgcggtaataaggtagtggtatcctatggtctaacgaaacaagagaaacaagacatcttaaaggagcacaatgactttagacaaaaaattgcacgaggattggagactagaggtaatcctggaccacagcctccagcgaagaatatgaaaaatttggtatggaacgacgagttagcttatgtcgcccaagtgtgggctaatcaatgtcaatatggtcacgatacttgcagggatgtagcaaaatatcaggttggacaaaacgtagccttaacaggtagcacggctgctaaatacgatgatccagttaaactagttaaaatgtgggaagatgaagtgaaagattataatcctaagaaaaagttttcgggaaacgactttctgaaaaccggccattacactcaaatggtttgggctaacaccaaggaagttggttgtggaagtataaaatacattcaagagaaatggcacaaacattaccttgtatgtaattatggacccagcggaaactttaagaatgaggaactttatcaaacaaagtaa

SEQ ID NO: 39 - protein, 204 aa, vespula vulgaris
NNYCKIKCLKGGVHTACKYGSLKPNCGNKVVVSYGLTKQEKQDILKEHNDFRQKIARGLETRGNPGPQPPAKNMKNLVWNDELAYVAQVWANQCQYGHDTCRDVAKYQVGQNVALTGSTAAKYDDPVKLVKMWEDEVKDYNPKKKFSGNDFLKTGHYTQMVWANTKEVGCGSIKYIQEKWHKHYLVCNYGPSGNFKNEELYQTK

SEQ ID NO: 40 - DNA, 38 bp, Artificial Sequence
  primer
ccgctcgagaaaagaaacaattattgtaaaataaaatg

(19) United States
(12) Patent Application Publication
(10) Pub. No.: US 2004/0091500 A1
(43) Pub. Date: May 13, 2004

(54) Recombinant allergens

(75) Inventors: Hans Henrik Ipsen, Hillerod (DK); Michael Dho Spangfort, Helsingborg (SE); Jorgen Nedergaard Larsen, Graested (DK)

Correspondence Address: DARBY & DARBY P.C., P. O. BOX 5257, NEW YORK, NY, 10150-5257, US

(73) Assignee: ALK-Abello A/S, Horsholm (DK)

(21) Appl. No.: 10/719,553

(22) Filed: Nov. 20, 2003

Related U.S. Application Data
(63) Continuation of application No. 09/270,910, filed on Mar. 16, 1999.
(60) Provisional application No. 60/078,371, filed on Mar. 18, 1998.

(30) Foreign Application Priority Data
Mar. 16, 1998 (DK) 0364/98

(51) Int. Cl. A61K 39/00; G06G 7/48; G06G 7/58

(52) U.S. Cl. 424/185.1; 703/11; 435/69.3

(57) ABSTRACT

Novel recombinant allergens are disclosed. The allergens are non-naturally occurring mutants derived from naturally-occurring allergens. The overall α-carbon backbone tertiary structure of the allergens is essentially preserved. Also disclosed are methods for preparing the recombinant allergens as well as the use of the recombinant allergens for the treatment of allergic reactions.

### CROSS REFERENCE TO RELATED APPLICATIONS

The present application is a continuation and claims the benefit of priority under 35 U.S.C. § 120 of Ser. No. 09/270,910, filed Mar. 16, 1999, and claims priority under 35 U.S.C. § 119(e) of provisional application No. 60/078,371, filed Mar. 18, 1998. Each of the foregoing applications is hereby incorporated by reference in its entirety.

### FIELD OF THE INVENTION

The present invention relates to novel recombinant allergens, which are non-naturally occurring mutants derived from naturally occurring allergens. Further, the invention relates to a method of preparing such recombinant allergens as well as to pharmaceutical compositions, including vaccines, comprising the recombinant allergens. In further embodiments, the present invention relates to methods of generating immune responses in a subject, vaccination or treatment of a subject as well as processes for preparing the compositions of the invention.

### BACKGROUND OF THE INVENTION

Genetically predisposed individuals become sensitised (allergic) to antigens originating from a variety of environmental sources, to the allergens of which the individuals are exposed. The allergic reaction occurs when a previously sensitised individual is re-exposed to the same or a homologous allergen. Allergic responses range from hay fever, rhinoconductivitis, rhinitis and asthma to systemic anaphylaxis and death in response to e.g. bee or hornet sting or insect bite. The reaction is immediate and can be caused by a variety of atopic allergens such as compounds originating from grasses, trees, weeds, insects, food, drugs, chemicals and perfumes.

However, the responses do not occur when an individual is exposed to an allergen for the first time. The initial adaptive response takes time and does usually not cause any symptoms. But when antibodies and T cells capable of reacting with the allergen have been produced, any subsequent exposure may provoke symptoms. Thus, allergic responses demonstrate that the immune response itself can cause significant pathological states, which may be life threatening.

The antibodies involved in atopic allergy belong primarily to immunoglobulins of the IgE class. IgE binds to specific receptors on the surface of mast cells and basophils. Following complex formation of a specific allergen with IgE bound to mast cells, receptor cross-linking on the cell surface results in signalling through the receptors and the physiological response of the target cells. Degranulation results in the release of i.a. histamine, heparin, a chemotactic factor for eosinophilic leukocytes, leukotrienes C4, D4 and E4, which cause prolonged constriction of the bronchial smooth muscle cells. The resulting effects may be systemic or local in nature.

The antibody-mediated hypersensitivity reactions can be divided into four classes, namely type I, type II, type III and type IV. Type I allergic reactions is the classic immediate hypersensitivity reaction occurring within seconds or minutes following antigen exposure. These symptoms are mediated by allergen specific IgE.

Commonly, allergic reactions are observed as a response to protein allergens present e.g. in pollens, house dust mites, animal hair and dandruff, venoms, and food products.

In order to reduce or eliminate allergic reactions, carefully controlled and repeated administration of allergy vaccines is commonly used. Allergy vaccination is traditionally performed by parenteral, intranasal, or sublingual administration in increasing doses over a fairly long period of time, and results in desensitisation of the patient. The exact immunological mechanism is not known, but induced differences in the phenotype of allergen specific T cells is thought to be of particular importance.

Antibody-Binding Epitopes (B-Cell Epitopes)

X-ray crystallographic analyses of F_{ab}-antigen complexes has increased the understanding of antibody-binding epitopes. According to this type of analysis antibody-binding epitopes can be defined as a section of the surface of the antigen comprising atoms from 15-25 amino acid residues, which are within a distance from the atoms of the antibody enabling direct interaction. The affinity of the antigen-antibody interaction can not be predicted from the enthalpy contributed by van der Waals interactions, hydrogen bonds or ionic bonds, alone. The entropy associated with the almost complete expulsion of water molecules from the interface represent an energy contribution similar in size. This means that perfect fit between the contours of the interacting molecules is a principal factor underlying antigen-antibody high affinity interactions.

Allergy Vaccination

The concept of vaccination is based on two fundamental characteristics of the immune system, namely specificity and memory. Vaccination will prime the immune system of the recipient, and upon repeated exposure to similar proteins the immune system will be in a position to respond more rigorously to the challenge of for example a microbial infection. Vaccines are mixtures of proteins intended to be used in vaccination for the purpose of generating such a protective immune response in the recipient. The protection will comprise only components present in the vaccine and homologous antigens.

Compared to other types of vaccination allergy vaccination is complicated by the existence of an ongoing immune response in allergic patients. This immune response is characterised by the presence of allergen specific IgE mediating the release of allergic symptoms upon exposure to allergens. Thus, allergy vaccination using allergens from natural sources has an inherent risk of side effects being in the utmost consequence life threatening to the patient.

Approaches to circumvent this problem may be divided in three categories. In practise measures from more than one category are often combined. First category of measures includes the administration of several small doses over prolonged time to reach a substantial accumulated dose. Second category of measures includes physical modification of the allergens by incorporation of the allergens into gel substances such as aluminium hydroxide. Aluminium hydroxide formulation has an adjuvant effect and a depot effect of slow allergen release reducing the tissue concentration of active allergen components. Third category of measures include chemical modification of the allergens for the purpose of reducing allergenicity, i.e. IgE binding.

The detailed mechanism behind successful allergy vaccination remains controversial. It is, however, agreed that T cells play a key role in the overall regulation of immune responses. According to current consensus the relation between two extremes of T cell phenotypes, Th1 and Th2, determine the allergic status of an individual. Upon stimulation with allergen Th1 cells secrete interleukines dominated by interferon-γ leading to protective immunity and the individual is healthy. Th2 cells on the other hand secrete predominantly interleukin 4 and 5 leading to IgE synthesis and eosinophilia and the individual is allergic. In vitro studies have indicated the possibility of altering the responses of allergen specific T cells by challenge with allergen derived peptides containing relevant T cell epitopes. Current approaches to new allergy vaccines are therefore largely based on addressing the T cells, the aim being to silence the T cells (anergy induction) or to shift the response from the Th2 phenotype to the Th1 phenotype.

In WO 97/30150 (ref. 1), a population of protein molecules is claimed, which protein molecules have a distribution of specific mutations in the amino acid sequence as compared to a parent protein. From the description, it appears that the invention is concerned with producing analogues which are modified as compared to the parent protein, but which are taken up, digested and presented to T cells in the same manner as the parent protein (naturally occurring allergens). Thereby, a modified T cell response is obtained. Libraries of modified proteins are prepared using a technique denoted PM (Parsimonious Mutagenesis).

In WO 92/02621 (ref. 2), recombinant DNA molecules are described, which molecules comprise a DNA coding for a polypeptide having at least one epitope of an allergen of trees of the order Fagales, the allergen being selected from Aln g 1, Cor a 1 and Bet v 1. The recombinant molecules described herein do all have an amino acid sequence or part of an amino acid sequence that corresponds to the sequence of a naturally occurring allergen.

WO 90/11293 (ref. 3) relates i.a. to isolated allergenic peptides of ragweed pollen and to modified ragweed pollen peptides. The peptides disclosed therein have an amino acid sequence corresponding either to the sequence of the naturally occurring allergen or to naturally occurring isoforms thereof.

Chemical Modification of Allergens

Several approaches to chemical modification of allergens have been taken. Approaches of the early seventies include chemical coupling of allergens to polymers, and chemical cross-linking of allergens using formaldehyde, etc., producing the so-called ‘allergoids’. The rationale behind these approaches was random destruction of IgE binding epitopes by attachment of the chemical ligand thereby reducing IgE-binding while retaining immunogenicity by the increased molecular weight of the complexes. Inherent disadvantages of ‘allergoid’ production are linked to difficulties in controlling the process of chemical cross-linking and difficulties in analysis and standardisation of the resulting high molecular weight complexes. ‘Allergoids’ are currently in clinical use and due to the random destruction of IgE binding epitopes higher doses can be administered as compared to conventional vaccines, but the safety and efficacy parameters are not improved over use of conventional vaccines.

More recent approaches to chemical modification of allergens aim at a total disruption of the tertiary structure of the allergen thus eliminating IgE binding assuming that the essential therapeutic target is the allergen specific T cell. Such vaccines contain allergen sequence derived synthetic peptides representing minimal T cells epitopes, longer peptides representing linked T cells epitopes, longer allergen sequence derived synthetic peptides representing regions of immunodominant T cell epitopes, or allergen molecules cut in two halves by recombinant technique. Another approach based on this rationale has been the proposal of the use of “low IgE binding” recombinant isoforms. In recent years it has become clear that natural allergens are heterogeneous containing isoallergens and variants having up to approximately 25% of their amino acids substituted. Some recombinant isoallergens have been found to be less efficient in IgE binding possibly due to irreversible denaturation and hence total disruption of tertiary structure.

In Vitro Mutagenesis and Allergy Vaccination

Attempts to reduce allergenicity by in vitro site directed mutagenesis have been performed using several allergens including Der f 2 (Takai et al, ref. 4), Der p 2 (Smith et al, ref. 5), a 39 kDa Dermatophagoides farinae allergen (Aki et al, ref. 6), bee venom phospholipase A2 (Förster et al, ref. 7), Ara h 1 (Burks et al, ref. 8), Ara h 2 (Stanley et al, ref. 9), Bet v 1 (Ferreira et al, ref. 10 and 11), birch profilin (Wiedemann et al, ref. 12), and Ory s 1 (Alvarez et al, ref. 13).

The rationale behind these approaches, again, is addressing allergen specific T cells while at the same time reducing the risk of IgE mediated side effects by reduction or elimination of IgE binding by disruption of the tertiary structure of the recombinant mutant allergen. The rationale behind these approaches does not include the concept of dominant IgE binding epitopes and it does not include the concept of initiating a new protective immune response which also involves B-cells and antibody generation.

The article by Ferreira et al (ref. 11) describes the use of site directed mutagenesis for the purpose of reducing IgE binding. Although the three-dimensional structure of Bet v 1 is mentioned in the article the authors do not use the structure for prediction of surface exposed amino acid residues for mutation, half of which have a low degree of solvent exposure. Rather they use a method developed for prediction of functional residues in proteins different from the concept of structure based identification of conserved surface areas described here. Although the authors do discuss conservation of α-carbon backbone tertiary structure this concept is not a part of the therapeutic strategy but merely included to assess in vitro IgE binding. Furthermore, the evidence presented is not adequate since normalisation of CD-spectra prevents the evaluation of denaturation of a proportion of the sample, which is a common problem. The therapeutic strategy described aim at inducing tolerance in allergen specific T cells and initiation of a new immune response is not mentioned.

The article by Wiedemann et al. (ref. 12) describes the use of site directed mutagenesis and peptide synthesis for the purpose of monoclonal antibody epitope characterisation. The authors have knowledge of the tertiary structure of the antigen and they use this knowledge to select a surface exposed amino acid for mutation. The algorithm used can be said to be opposite to the one described by the present inventors since an amino acid differing from homologous sequences is selected. The study demonstrates that substitution of a surface exposed amino acid has the capacity to modify the binding characteristics of a monoclonal antibody, which is not surprising considering common knowledge. The experiments described are not designed to assess modulation in the binding of polyclonal antibodies such as allergic patients' serum IgE. One of the experiments contained do apply serum IgE and although this experiment is not suitable for quantitative assessment, IgE binding does not seem to be affected by the mutations performed.

The article by Smith et al. (ref. 5) describes the use of site directed mutagenesis for the purpose of monoclonal antibody epitope mapping and reduction of IgE binding. The authors have no knowledge of the tertiary structure and make no attempt to assess the conservation of α-carbon backbone tertiary structure. The algorithm used does not ensure that amino acids selected for mutation are actually exposed to the molecular surface. Only one of the mutants described lead to a substantial reduction in IgE binding. This mutant is deficient in binding of all antibodies tested indicating that the tertiary structure is disrupted. The authors do not define a therapeutic strategy and initiation of a new immune response is not mentioned.

The article by Colombo et al. (ref. 14) describes the study of an IgE binding epitope by use of site directed mutagenesis and peptide synthesis. The authors use a three dimensional computer model structure based on the crystal structure of a homologous protein to illustrate the presence of the epitope on the molecular surface. The further presence of an epitope on a different allergen showing primary structure homology is addressed using synthetic peptides representing the epitope. The therapeutic strategy is based on treatment using this synthetic peptide representing a monovalent IgE binding epitope. Conserved surface areas between homologous allergens as well as the therapeutic concept of initiating a new protective immune response are not mentioned.

The article by Spangfort et al. (ref. 15) describes the three-dimensional structure and conserved surface exposed patches of the major birch allergen. The article does not mention major IgE binding epitopes nor site directed mutagenesis, neither is therapeutic application addressed.

In none of the studies described above is IgE binding reduced by substitution of surface exposed amino acids while conserving α-carbon backbone tertiary structure. The rationale behind above-mentioned approaches does not include the concept of dominant IgE binding epitopes and it does not include the therapeutic concept of initiating a new protective immune response.

### BRIEF DESCRIPTION OF THE FIGURES

FIG. 1 shows mutant-specific oligonucleotide primers used for Bet v 1 mutant number 1. Mutated nucleotides are underlined (SEQ ID NOS: 1-4).

FIG. 2 shows two generally applicable primers (denoted “all-sense” and “all non-sense”), which were synthesised and used for all mutants (SEQ ID NOS: 5-22).

FIG. 3 shows an overview of all Bet v 1 mutations (SEQ ID NOS: 36-37).

FIG. 4 shows the inhibition of the binding of biotinylated recombinant Bet v 1 to serum IgE from a pool of allergic patients by non-biotinylated Bet v 1 and by Bet v 1 Glu45Ser mutant.

FIG. 5 shows the inhibition of the binding of biotinylated recombinant Bet v 1 to serum IgE from a pool of allergic patients by non-biotinylated Bet v 1 and by Bet v 1 mutant Asn28Thr+Lys32Gln.

FIG. 6 shows the inhibition of the binding of biotinylated recombinant Bet v 1 to serum IgE from a pool of allergic patients by non-biotinylated Bet v 1 and by Bet v 1 Pro108Gly mutant.

FIG. 7 shows the inhibition of the binding of biotinylated recombinant Bet v 1 to serum IgE from a pool of allergic patients by non-biotinylated Bet v 1 and by Bet v 1 Glu60Ser mutant.

FIG. 8 shows the CD spectra of recombinant and Triple-patch mutant, recorded at close to equal concentrations.

FIG. 9 shows the inhibition of the binding of biotinylated recombinant Bet v 1 to serum IgE from a pool of allergic patients by non-biotinylated Bet v 1 and by Bet v 1 Triple-patch mutant.

FIG. 10 shows solvent accessibility of individually aligned antigen 5 residues and alignment of Vespula antigen 5 sequences (left panel). On the right panel of FIG. 10 is shown the molecular surface of antigen 5 with conserved areas among Vespula antigen 5:s.

FIG. 11 shows the sequence of the primer corresponding to the amino terminus of Ves v 5 derived from the sense strand (SEQ ID NOS: 23-30). The sequence of the downstream primer is derived from the non-sense strand.

FIG. 12 shows two generally applicable primers (denoted “all sense” and “all non-sense”, which were synthesised and used for all mutants (SEQ ID NOS: 31-35).

FIG. 13 shows an overview of all Ves v 5 mutations (SEQ ID NOS: 38-39).

FIG. 14 shows the inhibition of the binding of biotinylated recombinant Ves v 5 to serum IgE from a pool of allergic patients by non-biotinylated Ves v 5 and by Ves v 5 Lys72Ala mutant.

### OBJECT OF THE INVENTION

Rationale Behind the Present Invention

The current invention is based on a unique rationale. According to this rationale the mechanism of successful allergy vaccination is not an alteration of the ongoing Th2-type immune response, but rather a parallel initiation of a new Th1-type immune response involving tertiary epitope recognition by B-cells and antibody formation. This model is supported by the observation that levels of specific IgE are unaffected by successful vaccination treatment, and that successful treatment is often accompanied by a substantial rise in allergen specific IgG4. In addition, studies of nasal biopsies before and after allergen challenge do not show a reduction in T cells with the Th2-like phenotype, but rather an increase in Th1-like T cells are observed. When the vaccine (or pharmaceutical compositions) is administered through another route than the airways, it is hypothesised, that the new Th1-like immune response evolves in a location physically separated from the ongoing Th2 response thereby enabling the two responses to exist in parallel.

Another important aspect of the rationale behind the current invention is the assertion of the existence of dominant IgE binding epitopes. It is proposed that these dominant IgE binding epitopes are constituted by tertiary structure dependent coherent surface areas large enough to accommodate antibody binding and conserved among isoallergens, variants, and/or homologous allergens from related species. The existence of cross-reactive IgE capable of binding similar epitopes on homologous allergens is supported by the clinical observation that allergic patients often react to several closely related species, e.g. alder, birch (SEQ ID NOS: 36-37), and hazel, multiple grass species, or several species of the house dust mite genus Dermatophagoides. It is furthermore supported by laboratory experiments demonstrating IgE cross-reactivity between homologous allergens from related species and the capacity of one allergen to inhibit the binding of IgE to homologous allergens (Ipsen et al. 1992, ref. 16). It is well known that exposure and immune responses are related in a dose dependent fashion. Based on the combination of these observations it is hypothesised that conserved surface areas are exposed to the immune system in higher doses than non-conserved surface areas resulting in the generation of IgE antibodies with higher affinities, hence the term ‘dominant IgE binding epitopes’.

According to this rationale it is essential that the allergen has an α-carbon backbone tertiary structure which essentially is the same as that of the natural allergen, thus ensuring conservation of the surface topology of areas surrounding conserved patches representing targets for mutagenesis aimed at reducing IgE binding. By fulfilling these criteria the allergen has the potential to be administered in relatively higher doses improving its efficacy in generating a protective immune response without compromising safety.

### SUMMARY OF THE INVENTION

The present invention relates to the introduction of artificial amino acid substitutions into defined critical positions while retaining the α-carbon backbone tertiary structure of the allergen.

The invention provides a recombinant allergen, which is a non-naturally occurring mutant derived from a naturally occurring allergen, wherein at least one surface-exposed, conserved amino acid residue of a B cell epitope is substituted by another residue which does not occur in the same position in the amino acid sequence of any known homologous protein within the taxonomic order from which said naturally occurring allergen originates, said mutant allergen having essentially the same α-carbon backbone tertiary structure as said naturally occurring allergen, and the specific IgE binding to the mutated allergen being reduced as compared to the binding to said naturally occurring allergen.

Such recombinant allergen is obtainable by

a) identifying amino acid residues in a naturally occurring allergen which are conserved with more than 70% identity in all known homologous proteins within the taxonomic order from which said naturally occurring allergen originates;

b) defining at least one patch of conserved amino acid residues being coherently connected over at least 400 Å² of the surface of the three-dimensional of the allergen molecule as defined by having a solvent accessibility of at least 20%, said at least one patch comprising at least one B cell epitope; and

c) substituting at least one amino acid residue in said at least one patch by another amino acid being non-conservative in the particular position while essentially preserving the overall α-carbon backbone tertiary structure of the allergen molecule.

Specific IgE binding to the mutated allergen is preferably reduced by at least 5%, preferably at least 10% in comparison to naturally-occurring isoallergens or similar recombinant proteins in an immuno assay with sera from scource-specific IgE reactive allergic patients or pools thereof.

Recombinant allergens according to the invention may suitably be derived from inhalation allergens originating i.a. from trees, grasses, herbs, fungi, house dust mites, cockroaches and animal hair and dandruff. Important pollen allergens from trees, grasses and herbs are such originating from the taxonomic orders of Fagales, Oleales and Pinales including i.a. birch (Betula), alder (Alnus), hazel (Corylus), hornbeam (Carpinus) and olive (Olea), the order of Poales including i.a. grasses of the genera Lolium, Phelum, Poa, Cynodon, Dactylis and Secale, the orders of Asterales and Urticales including i.a. herbs of the genera Ambrosia and Artemisia. Important inhalation allergens from fungi are i.a. such originating from the genera Alternaria and Cladosporium. Other important inhalation allergens are those from house dust mites of the genus Dermatophagoides, those from cockroaches and those from mammals such as cat, dog and horse. Further, recombinant allergens according to the invention may be derived from venom allergens including such originating from stinging or biting insects such as those from the taxonomic order of Hymenoptera including bees (superfamily Apidae), wasps (superfamily Vespidea), and ants (superfamily Formicoidae).

Specific allergen components include e.g. Bet v 1 (B. verrucosa, birch), Aln g 1 (Alnus glutinosa, alder), Cor a 1 (Corylus avelana, hazel) and Car b 1 (Carpinus betulus, hornbeam) of the Fagales order. Others are Cry j 1 (Pinales), Amb a 1 and 2, Art v 1 (Asterales), Par j 1 (Urticales), Ole e 1 (Oleales), Ave e 1, Cyn d 1, Dac g 1, Fes p 1, Hol l 1, Lol p 1 and 5, Pas n 1, Phl p 1 and 5, Poa p 1, 2 and 5, Sec c 1 and 5, and Sor h 1 (various grass pollens), Alt a 1 and Cla h 1 (fungi), Der f 1 and 2, Der p 1 and 2 (house dust mites, D. farinae and D. pteronyssinus, respectively), Bla g 1 and 2, Per a 1 (cockroaches, Blatella germanica and Periplaneta americana, respectively), Fel d 1 (cat), Can f 1 (dog), Equ c 1, 2 and 3 (horse), Apis m 1 and 2 (honeybee), Ves g 1, 2 and 5, Pol a 1, 2 and 5 (all wasps) and Sol i 1, 2, 3 and 4 (fire ant).

In one embodiment, the recombinant allergen is derived from Bet v 1 (SEQ ID NOS: 36-37). Examples of substitutions are Thr10Pro, Asp25Gly, (Asn28Thr+Lys32Gln), Glu45Ser, Asn47Ser, Lys55Asn, Thr77Ala, Pro108Gly and (Asn28Thr, Lys32Gln, Glu45Ser, Pro108Gly. As apparent, the recombinant allergens may have one or more substitutions.

In another embodiment, the recombinant allergen is derived from a venom allergen from the taxonomic order of Vespidae, Apidae and Formicoidae.

In a further embodiment, the recombinant allergen is derived from Ves v 5 (SEQ ID NOS: 38-39). Examples of substitutions are Lys72Ala and Tyr96Ala. As apparent, the recombinant allergens may have one or more substitutions.

The present invention also provides a method of preparing a recombinant allergen as defined herein, comprising

a) identifying amino acid residues in a naturally occurring allergen which are conserved with more than 70% identity in all known homologous proteins within the taxonomic order from which said naturally occurring allergen originates;

b) defining at least one patch of conserved amino acid residues being coherently connected over at least 400 Å² of the surface of the three-dimensional structure of the allergen molecule as defined by having a solvent accessibility of at least 20%, said at least one patch comprising at least one B cell epitope, and

c) substituting at least one amino acid residue in said at least one patch by another amino acid being non-conservative in the particular position while essentially preserving the overall α-carbon backbone tertiary structure of the allergen molecule.

In this method the best results are obtained by ranking the amino acid residues of said at least one patch with respect to solvent accessibility and substituting one or more amino acids among the more solvent accessible ones.

Generally, in the method according to the invention the substitution of one or more amino acid residues in said B cell epitope or said at least one patch is carried out by site-directed mutagenesis.

Conservation of α-carbon backbone tertiary structure is best determined by obtaining identical structures by x-ray crystallography or NMR before and after mutagenesis. In absence of structural data describing the mutant indistinguishable CD-spectra or immunochemical data, e.g. antibody reactivity, may render conservation of α-carbon backbone tertiary structure probable, if compared to the data obtained by analysis of a structurally determined molecule.

Further, the present invention provides a pharmaceutical composition comprising a recombinant allergen as defined herein in combination with a pharmaceutically acceptable carrier and/or excipient, and optionally an adjuvant.

Such pharmaceutical composition may be in the form of a vaccine against allergic reactions elicited by a naturally occurring allergen in patients suffering from allergy.

In a further aspect, the present invention relates to a method of generating an immune response in a subject, which method comprises administering to the subject at least one recombinant allergen as defined herein, or a pharmaceutical composition comprising at least one recombinant allergen as defined herein.

The pharmaceutical composition of the invention can be prepared by a process comprising mixing at least one recombinant allergen as defined herein with pharmaceutically acceptable substances and/or excipients.

In a particular embodiment, the present invention concerns the vaccination or treatment of a subject, which vaccination of treatment comprises administering to the subject at least one recombinant allergen as defined herein or a pharmaceutical composition as defined herein.

The pharmaceutical compositions of the invention are obtainable by the process defined above.

In another embodiment, the recombinant allergens of the invention are suitable for use in a method for the treatment, prevention or alleviation of allergic reactions, such method comprising administering to a subject a recombinant allergen as defined herein or a pharmaceutical composition as defined herein.

### DETAILED DESCRIPTION OF THE INVENTION

Criteria for Substitution

For molecules for which the tertiary structure has been determined (e.g. by x-ray crystallography, or NMR electron microscopy), the mutant carrying the substituted amino acid(s) should preferably fulfil the following criteria:

1. The overall α-carbon backbone tertiary structure of the molecule should be conserved. Conserved is defined as an average root mean square deviation of the atomic coordinates comparing the structures below 2 Å. This is important for two reasons: a) It is anticipated that the entire surface of the natural allergen constitutes an overlapping continuum of potential antibody-binding epitopes. The majority of the surface of the molecule is not affected by the substitution(s), and thus retain its antibody-binding properties, which is important for the generation of new protective antibody specificities being directed at epitopes present also on the natural allergen. b) Stability, both concerning shelf-life and upon injection into body fluids.

2. The amino acid(s) to be substituted should be located at the surface, and thus be accessible for antibody-binding. Amino acids located on the surface are defined as amino acids in the three-dimensional structure having a solvent (water) accessibility of at least 20%, suitably 20-80%, more suitably 30-80%. Solvent accessibility is defined as the area of the molecule accessible to a sphere with a radius comparable to a solvent (water, r=1.4 Å) molecule.

3. The substituted amino acid(s) should be located in conserved patches larger than 400 Å². Conserved patches are defined as coherently connected areas of surface exposed conserved amino acid residues and backbone. Conserved amino acid residues are defined by sequence alignment of all known (deduced) amino acid sequences of homologues proteins within the taxonomical order. Amino acid positions having identical amino acid residues in more than 90% of the sequences are considered conserved. Conserved patches are expected to contain epitopes to which the IgE of the majority of patients is directed.

4. Within the conserved patches amino acids for mutagenesis should preferentially be selected among the most solvent (water) accessible ones located preferably near the centre of the conserved patch.

Preferentially, a polar amino acid residue is substituted by another polar residue, and a non-polar amino acid residue is substituted by another non-polar residue.

Preparation of vaccines is generally well-known in the art. Vaccines are typically prepared as injectables either as liquid solutions or suspensions. Such vaccine may also be emulsified or formulated so as to enable nasal administration. The immunogenic component in question (the recombinant allergen as defined herein) may suitably be mixed with excipients which are pharmaceutically acceptable and further compatible with the active ingredient. Examples of suitable excipients are water, saline, dextrose, glycerol, ethanol and the like as well as combinations thereof. The vaccine may additionally contain other substances such as wetting agents, emulsifying agents, buffering agents or adjuvants enhancing the effectiveness of the vaccine.

Vaccines are most frequently administered parenterally by subcutaneous or intramuscular injection. Formulations which are suitable for administration by another route include oral formulations and suppositories. Vaccines for oral administration may suitably be formulated with excipients normally employed for such formulations, e.g. pharmaceutical grades of mannitol, lactose, starch, magnesium stearate, sodium saccharine, cellulose, magnesium carbonate and the like. The composition can be formulated as solutions, suspensions; emulsions, tablets, pills, capsules, sustained release formulations, aerosols, powders, or granulates.

The vaccines are administered in a way so as to be compatible with the dosage formulation and in such amount as will be therapeutically effective and immunogenic. The quantity of active component contained within the vaccine depends on the subject to be treated, i.a. the capability of the subject's immune system to respond to the treatment, the route of administration and the age and weight of the subject. Suitable dosage ranges can vary within the range from about 0.0001 μg to 1000 μg.

As mentioned above, an increased effect may be obtained by adding adjuvants to the formulation. Examples of such adjuvants are aluminum hydroxide and phosphate (alum) as a 0.05 to 0.1 percent solution in phosphate buffered saline, synthetic polymers of sugars used as 0.25 percent solution. Mixture with bacterial cells such as C. parvum, endotoxins or lipopolysaccharide components of gram-negative bacteria, emulsion in physiologically acceptable oil vehicles such as mannide monoaleate (Aracel A) or emulsion with 20 percent solution of a perfluorocarbon (e.g. Fluosol-DA) used as a block substitute may also be employed. Other adjuvants such as Freund's complete and incomplete adjuvants as well as QuilA and RIBI may also be used.

Most often, multiple administrations of the vaccine will be necessary to ensure an effect. Frequently, the vaccine is administered as an initial administration followed by subsequent inoculations or other administrations. The number of vaccinations will typically be in the range of from 1 to 50, usually not exceeding 35 vaccinations. Vaccination will normally be performed from biweekly to bimonthly for a period of 3 months to 5 years. This is contemplated to give desired level of prophylactic or therapeutic effect.

The recombinant allergen may be used as a pharmaceutical preparation, which is suitable for providing a certain protection against allergic responses during the period of the year where symptoms occur (prophylaxis). Usually, the treatment will have to be repeated every year to maintain the protective effect. Preparations formulated for nasal application are particular suited for this purpose.

The present invention is further illustrated by the following non-limiting examples.

### EXAMPLES

### Example 1

Identification of Common Epitopes within Fagales Pollen Allergens

The major birch pollen allergen Bet v 1 (SEQ ID NO: 37) shows about 90% amino acid sequence identity with major allergens from pollens of taxonomically related trees, i.e Fagales (for instance hazel and hornbeam) and birch pollen allergic patients often show clinical symptoms of allergic cross-reactivity towards these Bet v 1 homologous proteins.

Bet v 1 (SEQ ID NO: 37) also shows about 50-60% sequence identity with allergic proteins present in certain fruits (for instance apple and cherry) and vegetables (for instance celery and carrot) and there are clinical evidence for allergic cross-reactivity between Bet v 1 (SEQ ID NO: 37) and these food related proteins.

In addition, Bet v 1 shares significant sequence identity (20-40%) with a group of plant proteins called pathogenesis-related proteins (PR-10), however there are no reports of allergic cross-reactivity towards these PR-10 proteins.

Molecular modelling suggests that the structures of Fagales and food allergens and PR-10 proteins are close to be identical with the Bet v 1 (SEQ ID NO: 37) structure.

The structural basis for allergic Bet v 1 (SEQ ID NO: 37) cross-reactivity was reported in (Gajhede et al 1996, ref. 17) where three patches on the molecular surface of Bet v 1 (SEQ ID NO: 37) could be identified to be common for the known major tree pollen allergens. Thus, any IgE recognising these patches on Bet v 1 (SEQ ID NO: 37) would be able to cross-react and bind to other Fagales major pollen allergens and give rise to allergic symptoms. The identification of these common patches was performed after alignment of all known amino acid sequences of the major tree pollen allergens in combination with an analysis of the molecular surface of Bet v 1 (SEQ ID NO: 37) revealed by the α-carbon backbone tertiary structure reported in ref. 17. In addition, the patches were defined to have a certain minimum size (>400 Å²) based on the area covered by an antibody upon binding.

Selection of Amino Acid Residues for Site-Directed Mutagenesis

Amino acid residues for site-directed mutagenesis were selected among residues present in Bet v 1 (SEQ ID NO: 37) specific areas and the common patches since modifications of these is expected to affect the binding of serum IgE from the majority of patients showing clinical tree pollen allergic cross-reactivity.

The relative orientation and percentage of solvent-exposure of each amino acid residue within respective patch was calculated based on their atomic coordinates. Residues having a low degree of solvent exposure (<20%) were not regarded relevant for mutagenesis due to the possible disruption of the structure or lack of antibody interaction. The remaining residues were ranked according to their degree of solvent-exposure.

Sequence Alignment

Sequences homologous to the query sequence (Bet v 1 No. 2801, WHO IUIS Nomenclature Subcommittee on Allergens (SEQ ID NOS: 36-37) were derived from GenBank and EMBL sequence databases by a BLAST search (Altschul et al., ref. 18). All sequences with BLAST reported probabilities less than 0.1 were taken into consideration and one list were constructed containing a non-redundant list of homologous sequences. These were aligned by CLUSTAL W (Higgins et al., ref. 19) and the percentage identity were calculated for each position in the sequence considering the complete list or taxonomically related species only. A total of 122 sequences were homologous to Bet v 1 No. 2801 (SEQ ID NOS: 36-37) of which 57 sequences originates from taxonomically related species.

Cloning of the gene encoding Bet v 1 (SEQ ID NO: 36)

RNA was prepared from Betula verrucosa pollen (Allergon, Sweden) by phenol extraction and LiCl precipitation. Oligo(dT)-cellulose affinity chromatography was performed batch-wise in Eppendorph tubes, and double-stranded cDNA was synthesised using a commercially available kit (Amersham). DNA encoding Bet v 1 (SEQ ID NO: 36) was amplified by PCR and cloned. In brief, PCR was performed using cDNA as template, and primers designed to match the sequence of the cDNA in positions corresponding to the amino terminus of Bet v 1 (SEQ ID NO: 37) and the 3′-untranslated region, respectively. The primers were extended in the 5′-ends to accommodate restriction sites (NcoI and HindIII) for directional cloning into pKK233-2.

Subcloning into pMAL-c

The gene encoding Bet v 1 (SEQ ID NO: 36) was subsequently subcloned into the maltose binding protein fusion vector pMAL-c (New England Biolabs). The gene was amplified by PCR and subcloned in frame with malE to generate maltose binding protein (MBP)-Bet v 1 (SEQ ID NO: 37) protein fusion operons in which MBP and Bet v 1 were separated by a factor Xₐ protease clevage site positioned to restore the authentic aminoterminal sequence of Bet v 1 (SEQ ID NO: 37) upon cleavage, as described in ref. 15. In brief, PCR was performed using pKK233-3 with Bet v 1 (SEQ ID NO: 36) inserted as template and primers corresponding to the amino- and carboxyterminus of the protein, respectively. The promoter proximal primer was extended in the 5′-end to accommodate 4 codons encoding an in frame factor Xₐ protease cleavage site. Both primers were furthermore extended in the 5′-ends to accommodate restriction sites (KpnI) for cloning. The Bet v 1 encoding genes were subcloned using 20 cycles of PCR to reduce the frequency of PCR artefacts.

In Vitro Mutagenesis

In vitro mutagenesis was performed by PCR using recombinant pMAL-c with Bet v 1 inserted as template. Each mutant Bet v 1 gene was generated by 3 PCR reactions using 4 primers.

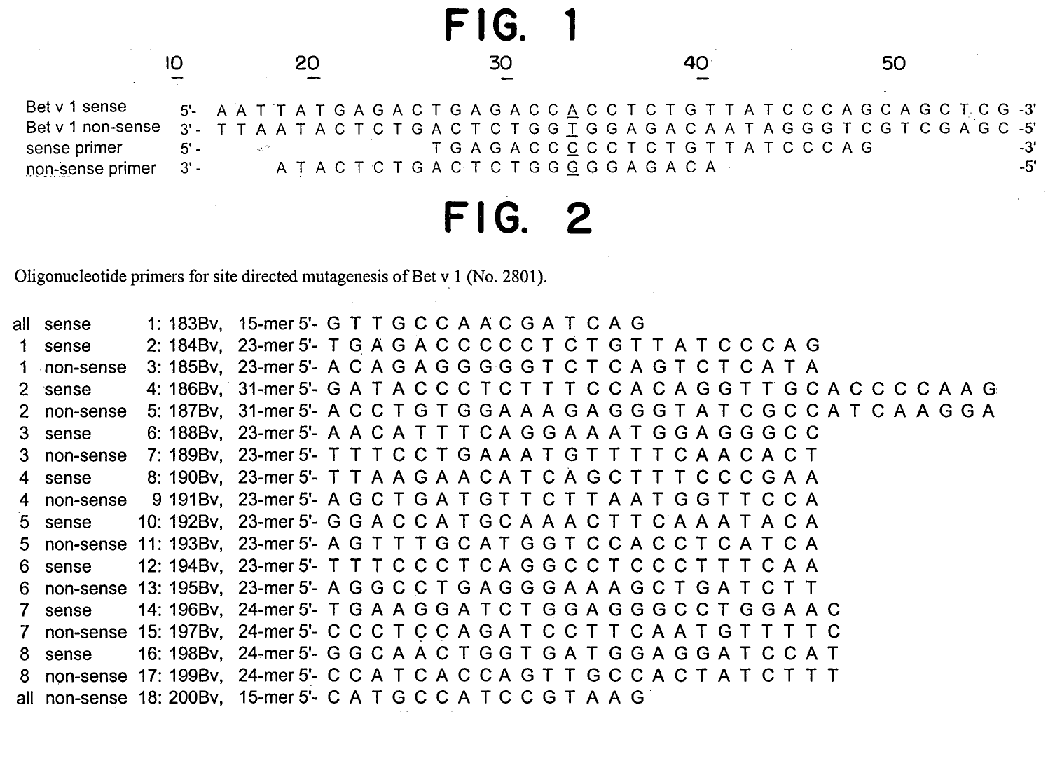

Two mutation-specific oligonucleotide primers were synthesised accommodating each mutation, one for each DNA strand, see FIGS. 1 and 2, Using the mutated nucleotide(s) as starting point both primers were extended 7 nucleotides in the 5′-end and 15 nucleotides in the 3′-end. The extending nucleotides were identical in sequence to the Bet v 1 gene in the actual region.

Two generally applicable primers (denoted “all-sense” and “all non-sense” in FIG. 2) were furthermore synthesised and used for all mutants. These primers were 15 nucleotides in length and correspond in sequence to regions of the pMAL-c vector approximately 1 kilobase upstream and downstream from the Bet v 1. The sequence of the upstream primer is derived from the sense strand and the sequence of the downstream primer is derived from the non-sense strand, see FIG. 2.

Two independent PCR reactions were performed essentially according to standard procedures (Saiki et al 1988, ref. 20) with the exception that only 20 temperature cycles were performed in order to reduce the frequency of PCR artefacts. Each PCR reaction used pMAL-c with Bet v 1 inserted as template and one mutation-specific and one generally applicable primer in meaningful combinations.

Introduction of the four amino acid substitutions (Asn28Thr, Lys32Gln, Glu45Ser, Pro108Gly) in the Triple-patch mutant were performed like described above in a step by step process (SEQ ID NO: 37). First the Glu45Ser mutation then the Pro108Gly mutation and last the Asn28Thr, Lys32Gln mutations were introduced using pMAL-c with inserted Bet v 1 No. 2801, Bet v 1 (Glu45Ser), Bet v 1 (Glu45Ser, Pro108Gly) as templates, respectively (SEQ ID NO: 37).

The PCR products were purified by agarose gel electrophoresis and electro-elution followed by ethanol precipitation. A third PCR reaction was performed using the combined PCR products from the first two PCR reactions as template and both generally applicable primers. Again, 20 cycles of standard PCR were used. The PCR product was purified by agarose gel electrophoresis and electro-elution followed by ethanol precipitation, cut with restriction enzymes (BsiWI/EcoRI), and ligated directionally into pMAL-c with Bet v 1 (SEQ ID NO: 36) inserted restricted with the same enzymes.

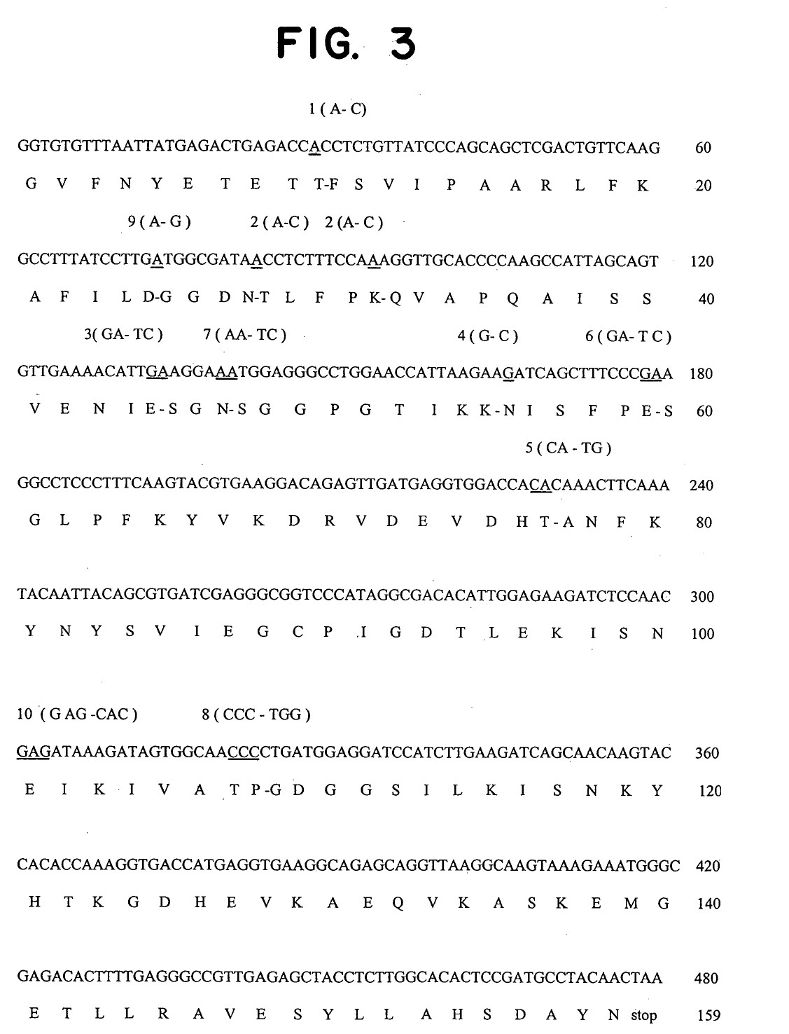

FIG. 3 shows an overview of all 9 Bet v 1 mutations, which are as follows (SEQ ID NOS: 36-37):

Thr10Pro, Asp25Gly, Asn28Thr+Lys32Gln, Glu45Ser, Asn47Ser, Lys55Asn, Glu60Ser (non-patch), Thr77Ala and Pro108Gly (SEQ ID NO: 37). An additional four mutant with four mutations was also prepared (Asn28Thr, Lys32Gln, Glu45Ser, Pro108Gly) (SEQ ID NO: 37). Of these, five were selected for further testing: Asn28Thr+Lys32Gln, Glu45Ser, Glu60Ser, Pro108Gly and the Triple-patch mutant Asn28Thr, Lys32Gln, Glu45Ser, Pro108Gly (SEQ ID NO: 37).

Nucleotide Sequencing

Determination of the nucleotide sequence of the Bet v 1 encoding gene (SEQ ID NO: 36) was performed before and after subcloning, and following in vitro mutagenesis, respectively.

Plasmid DNA's from 10 ml of bacterial culture grown to saturation overnight in LB medium supplemented with 0.1 g/l ampicillin were purified on Qiagen-tip 20 columns and sequenced using the Sequenase version 2.0 DNA sequencing kit (USB) following the recommendations of the suppliers.

Expression and Purification of Recombinant Bet v 1 and Mutants

Recombinant Bet v 1 (Bet v 1 No. 2801 and mutants (SEQ ID NO: 37) were over-expressed in Escherichia coli DH 5a fused to maltose-binding protein and purified as described in ref. 15. Briefly, recombinant E. coli cells were grown at 37° C. to an optical density of 1.0 at 436 nm, whereupon expression of the Bet v 1 fusion protein was induced by addition of IPTG. Cells were harvested by centrifugation 3 hours post-induction, re-suspended in lysis buffer and broken by sonication. After sonication and additional centrifugation, recombinant fusion protein was isolated by amylose affinity chromatography and subsequently cleaved by incubation with Factor Xa (ref. 15). After F Xa cleavage, recombinant Bet v 1 (SEQ ID NO: 37) was isolated by gelfiltration and if found necessary, subjected to another round of amylose affinity chromatography in order to remove trace amounts of maltose-binding protein.

Purified recombinant Bet v 1 (SEQ ID NO: 37) was concentrated by ultrafiltration to about 5 mg/ml and stored at 4° C. The final yields of the purified recombinant Bet v 1 (SEQ ID NO: 37) preparations were between 2-5 mg per litre E. coli cell culture.

The purified recombinant Bet v 1 (SEQ ID NO: 37) preparations appeared as single bands after silver-stained SDS-polyacrylamide electrophoresis with an apparent molecular weight of 17.5 kDa. N-terminal sequencing showed the expected sequences as derived from the cDNA nucleotide sequences and quantitative amino acid analysis showed the expected amino acid compositions.

We have previously shown (ref. 15) that recombinant Bet v 1 No. 2801 (SEQ ID NO: 37) is immunochemically indistinguishable from naturally occurring Bet v 1.

Immunoelectrophoresis Using Rabbit Polyclonal Antibodies

The seven mutant Bet v 1 (SEQ ID NO: 37) were produced as recombinant Bet v 1 proteins (SEQ ID NO: 37) and purified as described above and tested for their reactivity towards polyclonal rabbit antibodies raised against Bet v 1 (SEQ ID NO: 37) isolated from birch pollen. When analysed by immunoelectrophoresis (rocket-line immunoelectrophoresis) under native conditions, the rabbit antibodies were able to precipitate all mutants, indicating that the mutants had conserved α-carbon backbone tertiary structure.

These results suggested that non-naturally occurring substitutions introduced on the molecular surface of Bet v 1 (SEQ ID NO: 37) can reduce a polyclonal antibody response raised against naturally occurring Bet v 1 (SEQ ID NO: 37) without distortion of the overall α-carbon backbone tertiary allergen structure. In order to analyse the effect on human polyclonal IgE-response, the mutants Glu45Ser, Pro108Gly, Asn28Thr+Lys32Gln and Glu60Ser were selected for further analysis (SEQ ID NO: 37).

Bet v 1 Glu45Ser Mutant (SEQ ID NO: 37)

Glutamic acid in position 45 show a high degree of solvent-exposure (40%) and is located in a molecular surface patch common for Fagales allergens (patch I). A serine residue was found to occupy position 45 in some of the Bet v 1 (SEQ ID NO: 37) homologous PR-10 proteins arguing for that glutamic acid can be replaced by serine without distortion of the α-carbon backbone tertiary structure. In addition, as none of the known Fagales allergen sequences have serine in position 45, the substitution of glutamic acid with serine gives rise to a non-naturally occurring Bet v 1 molecule (SEQ ID NO: 37).

T Cell Proliferation Assay Using Recombinant Glu45Ser Bet v 1 Mutant (SEQ ID NO: 37)

The analysis was carried out as described in Spangfort et al 1996a. It was found that recombinant Bet v 1 Glu45Ser mutant was able to induce proliferation in T cell lines from three different birch pollen allergic patients with stimulation indices similar to recombinant and naturally occurring.

Crystallisation and Structural Determination of Recombinant Glu45Ser Bet v 1 (SEQ ID NO: 37)

Crystals of recombinant Glu45Ser Bet v 1 (SEQ ID NO: 37) were grown by vapour diffusion at 25° C., essentially as described in (Spangfort et al 1996b, ref. 21). Glu45Ser Bet v 1 (SEQ ID NO: 37), at a concentration of 5 mg/ml, was mixed with an equal volume of 2.0 M ammonium sulphate, 0.1 M sodium citrate, 1% (v/v) dioxane, pH 6.0 and equilibrated against 100× volume of 2.0 M ammonium sulfate, 0.1 M sodium citrate, 1% (v/v) dioxane, pH 6.0. After 24 hours of equilibration, crystal growth was induced by applying the seeding technique described in ref. 21, using crystals of recombinant wild-type Bet v 1 (SEQ ID NO: 37) as a source of seeds.

After about 2 months, crystals were harvested and analysed using X-rays generated from a Rigaku rotating anode as described in ref. 21 and the structure was solved using molecular replacement.

Structure of Bet v 1 Glu45Ser Mutant (SEQ ID NO: 37)

The structural effect of the mutation was addressed by growing three-dimensional Bet v 1 Glu45Ser protein (SEQ ID NO: 37) crystals diffracting to 3.0 Å resolution when analysed by X-rays generated from a rotating anode. The substitution of glutamic acid to serine in position 45 was verified by the Bet v 1 Glu45Ser (SEQ ID NO: 37) structure electron density map which also showed that the overall α-carbon backbone tertiary structure is preserved.

IgE-Binding Properties of Bet v 1 Glu45Ser Mutant (SEQ ID NO: 37)

The IgE-binding properties of Bet v 1 Glu45Ser mutant (SEQ ID NO: 37) was compared with recombinant Bet v 1 in a fluid-phase IgE-inhibition assay using a pool of serum IgE derived from birch allergic patients.

Recombinant Bet v 1 no. 2801 (SEQ ID NO: 37) was biotinylated at a molar ratio of 1:5 (Bet v 1 no. 2801:biotin). The inhibition assay was performed as follows: a serum sample (25 μl) was incubated with solid phase anti IgE, washed, re-suspended and further incubated with a mixture of biotinylated Bet v 1 no. 2801 (SEQ ID NO: 37) (3.4 nM) and a given mutant (0-28.6 nM). The amount of biotinylated Bet v 1 no. 2801 (SEQ ID NO: 37) bound to the solid phase was estimated from the measured RLU after incubation with acridinium ester labelled streptavidin. The degree of inhibition was calculated as the ratio between the RLU's obtained using buffer and mutant as inhibitor.

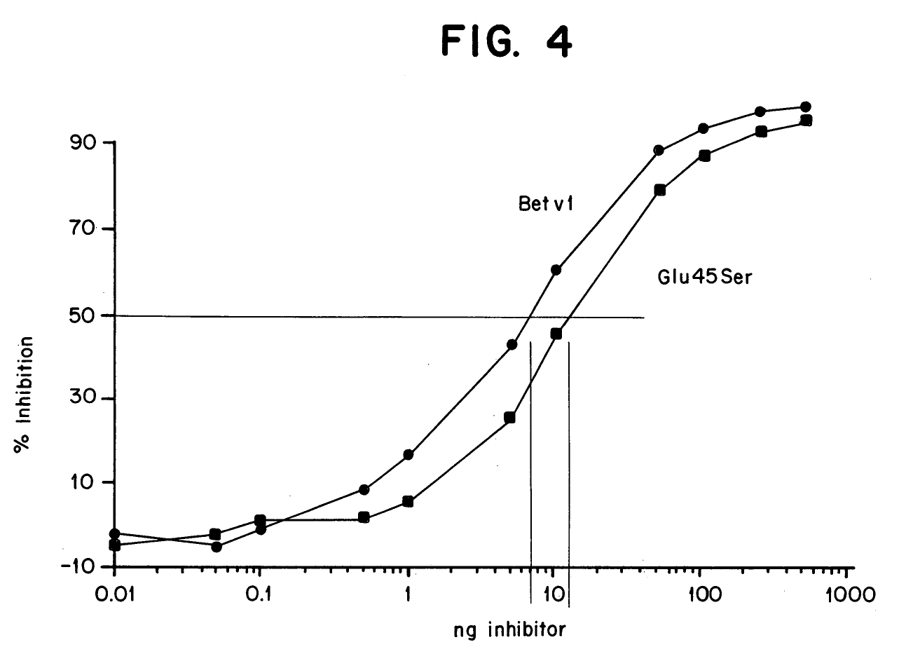

FIG. 4 shows the inhibition of the binding of biotinylated recombinant Bet v 1 (SEQ ID NO: 37) to serum IgE from a pool of allergic patients by non-biotinylated Bet v 1 and by Bet v 1 Glu45Ser mutant (SEQ ID NO: 37).

There is a clear difference in the amount of respective recombinant proteins necessary to reach 50% inhibition of the binding to serum IgE present in the serum pool. Recombinant Bet v 1 (SEQ ID NO: 37) reaches 50% inhibition at about 6.5 ng whereas the corresponding concentration for Bet v 1 Glu45Ser mutant (SEQ ID NO: 37) is about 12 ng. This show that the point mutation introduced in Bet v 1 Glu45Ser mutant (SEQ ID NO: 37) lowers the affinity for specific serum IgE by a factor of about 2.

The maximum level of inhibition reached by the Bet v 1 Glu45Ser mutant (SEQ ID NO: 37) is clearly lower compared to recombinant Bet v 1. (SEQ ID NO: 37) This may indicate that after the Glu45Ser substitution (SEQ ID NO: 37), some of the specific IgE present in the serum pool are unable to recognise the Bet v 1 Glu45Ser mutant (SEQ ID NO: 37).

Bet v 1 Mutant Asn28Thr+Lys32Gln (SEQ ID NO: 37)

Aspartate and lysine in positions 28 and 32, respectively show a high degree of solvent-exposure (35% and 50%, respectively) and are located in a molecular surface patch common for Fagales allergens (patch II). In the structure, aspartate 28 and lysine 32 are located close to each other on the molecular surface and most likely interact via hydrogen bonds. A threonine and a gluatamate residue were found to occupy positions 28 and 32, respectively in some of the Bet v 1 homologous PR-10 proteins arguing for that aspartate and lysine can be replaced with threonine and glutamate, respectively without distortion of the α-carbon backbone tertiary structure. In addition, as none of the naturally occurring isoallergen sequences have threonine and glutamate in positions 28 and 32, respectively, the substitutions gives rise to a non-naturally occurring Bet v 1 molecule (SEQ ID NO: 37).

IgE-Binding Properties of Bet v 1 Mutant Asn28Thr+Lys32Gln (SEQ ID NO: 37)

The IgE-binding properties of mutant Asn28Thr+Lys32Gln (SEQ ID NO: 37) was compared with recombinant Bet v 1 in a fluid-phase IgE-inhibition assay using the pool of serum IgE derived from birch allergic patients described above.

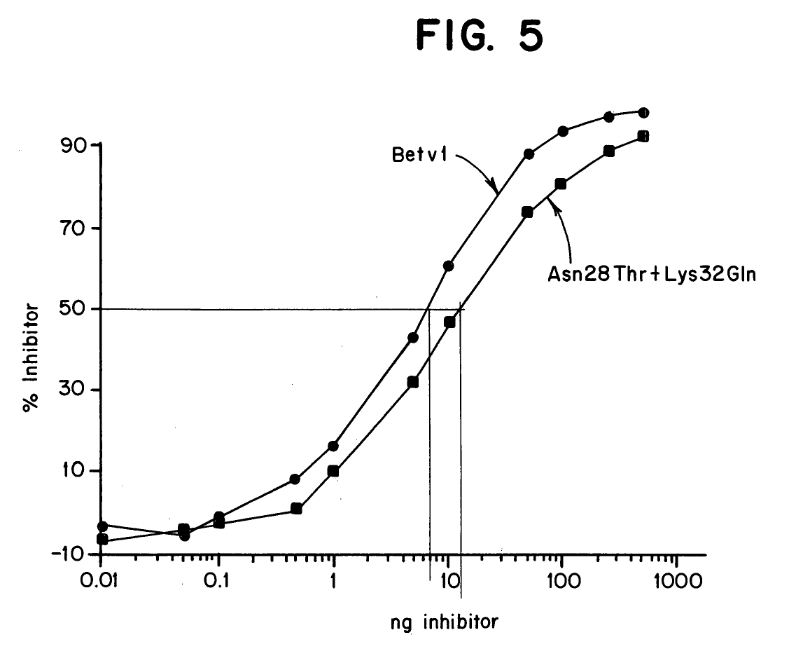

FIG. 5 shows the inhibition of the binding of biotinylated recombinant Bet v 1 (SEQ ID NO: 37) to serum IgE from a pool of Allergic patients by non-biotinylated Bet v 1 and by Bet v 1 mutant Asn28Thr+Lys32Gln (SEQ ID NO: 37).

There is a clear difference in the amount of respective recombinant proteins necessary to reach 50% inhibition of the binding to serum IgE present in the serum pool. Recombinant Bet v 1 reaches 50% inhibition at about 6.5 ng whereas the corresponding concentration for Bet v 1 mutant Asn28Thr+Lys32Gln (SEQ ID NO: 37) is about 12 ng. This show that the point mutations introduced in Bet v 1 (SEQ ID NO: 37) mutant Asn28Thr+Lys32Gln (SEQ ID NO: 37) lowers the affinity for specific serum IgE by a factor of about 2.

The maximum level of inhibition reached by the Bet v 1 mutant Asn28Thr+Lys32Gln mutant (SEQ ID NO: 37) is clearly lower compared to recombinant Bet v 1. This may indicate that after the Asn28Thr+Lys32Gln substitutions, some of the specific IgE present in the serum pool are unable to recognise the Bet v 1 mutant Asn28Thr+Lys32Gln (SEQ ID NO: 37).

Bet v 1 Mutant Pro108Gly (SEQ ID NO: 37)

Proline in position 108 show a high degree of solvent-exposure (60%) and is located in a molecular surface patch common for Fagales allergens (patch III). A glycine residue was found to occupy position 108 in some of the Bet v 1 homologous PR-10 proteins arguing for that proline can be replaced with glycine without distortion of the α-carbon backbone tertiary structure. In addition, as none of the naturally occurring isoallergen sequences have glycine in position 108, the substitution of proline with glycine gives rise to a non-naturally occurring Bet v 1 molecule (SEQ ID NO: 37).

IgE-Binding Properties of Bet v 1 Pro108Gly Mutant (SEQ ID NO: 37)

The IgE-binding properties of Bet v 1 Pro108Gly mutant (SEQ ID NO: 37) was compared with recombinant Bet v 1 (SEQ ID NO: 37) in a fluid-phase IgE-inhibition assay using the pool of serum IgE derived from birch allergic patients described above.

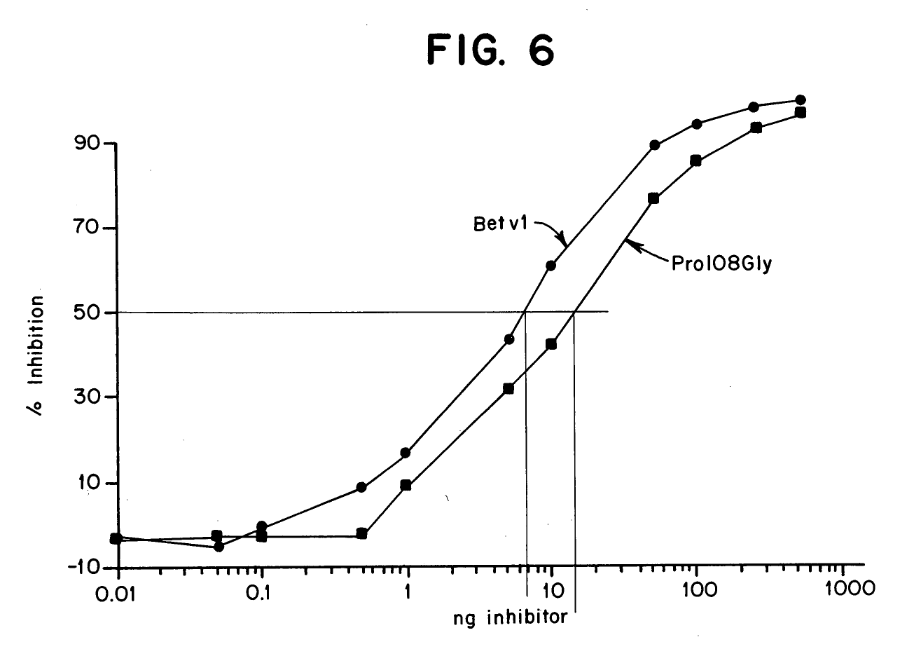

FIG. 6 shows the inhibition of the binding of biotinylated recombinant Bet v 1 (SEQ ID NO: 37) to serum IgE from a pool of allergic patients by non-biotinylated Bet v 1 (SEQ ID NO: 37) and by Bet v 1 Pro108Gly mutant (SEQ ID NO: 37).

There is a clear difference in the amount of respective recombinant proteins necessary to reach 50% inhibition of the binding to serum IgE present in the serum pool. Recombinant Bet v 1 reaches 50% inhibition at about 6.5 ng whereas the corresponding concentration for Bet v 1 Pro108Gly (SEQ ID NO: 37) is 15 ng. This show that the single point mutation introduced in Bet v 1 Pro108Gly (SEQ ID NO: 37) lowers the affinity for specific serum IgE by a factor of about 2.

The maximum level of inhibition reached by the Bet v 1 Pro108Gly mutant (SEQ ID NO: 37) is somewhat lower compared to recombinant Bet v 1 (SEQ ID NO: 37). This may indicate that after the Pro108Gly substitution, some of the specific IgE present in the serum pool are unable to recognise the Bet v 1 Pro108Gly mutant (SEQ ID NO: 37).

Bet v 1 Mutant Glu60Ser (Non-Patch Mutant) (SEQ ID NO: 37)

Glutamic acid in position 60 show a high degree of solvent-exposure (60%) however, it is not located in a molecular surface patch common for Fagales allergens. A serine residue was found to occupy position 60 in some of the Bet v 1 homologous PR-10 proteins arguing for that glutamic acid can be replaced with serine without distortion of the α-carbon backbone tertiary structure. In addition, as none of the naturally occurring isoallergen sequences have serine in position 60, the substitution of glutamic acid with serine gives rise to a non-naturally occurring Bet v 1 molecule (SEQ ID NO: 37).

IgE-Binding Properties of Bet v 1 Glu60Ser Mutant (SEQ ID NO: 37)

The IgE-binding properties of Bet v 1 Glu60Ser mutant (SEQ ID NO: 37) was compared with recombinant Bet v 1 in a fluid-phase IgE-inhibition assay using the pool of serum IgE derived from birch allergic patients described above.

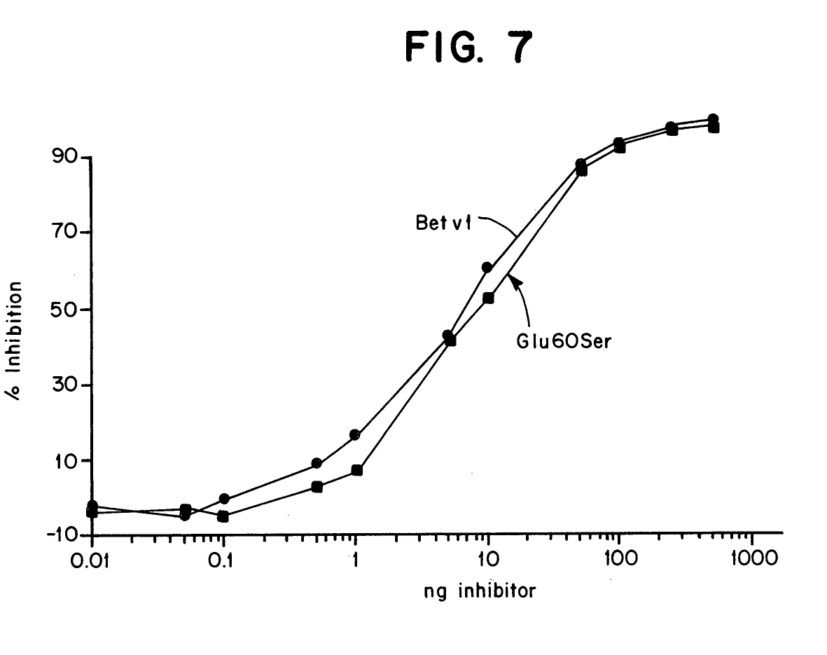

FIG. 7 shows the inhibition of the binding of biotinylated recombinant Bet v 1 (SEQ ID NO: 37) to serum IgE from a pool of allergic patients by non-biotinylated Bet v1 (SEQ ID NO: 37) and by Bet v1 Glu60Ser mutant (SEQ ID NO: 37). In contrast to the Glu45Ser, Pro108Gly and Asn28Thr+Lys32Gln mutants (SEQ ID NO: 37), the substitution glutamic acid 60 to serine, does not show any significant effect on the IgE-binding properties of Bet v 1 Glu60Ser mutant (SEQ ID NO: 37). This indicates that substitutions outside the defined Fagales common patches only have a marginal effect on the binding of specific serum IgE supporting the concept that conserved allergen molecular surface areas harbor dominant IgE-binding epitopes.

Bet v 1 Triple-Patch Mutant (SEQ ID NO: 37)

In the Triple-patch mutant, the point mutations (Glu45Ser, Asn28Thr+Lys32Gln and Pro108Gly) introduced in the three different common Fagales patches, described above, were simultaneously introduced in creating an artificial mutant carrying four amino acid substitutions.

Structural Analysis of Bet v 1 Triple-Patch Mutant (SEQ ID NO: 37)

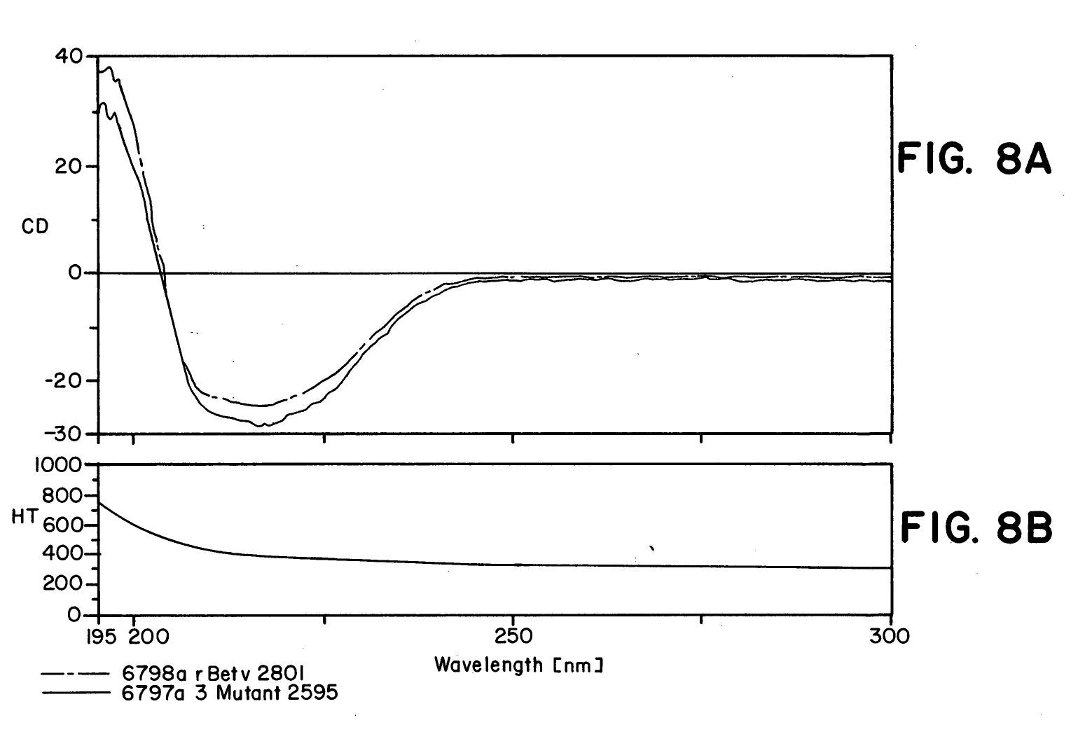

The structural integrity of the purified Triple-patch mutant was analysed by circular dichroism (CD) spectroscopy. FIG. 8 shows the CD spectra of recombinant and Triple-patch mutant (SEQ ID NO: 37), recorded at close to equal concentrations. The overlap in peak amplitudes and positions in the CD spectra from the two recombinant proteins shows that the two preparations contain equal amounts of secondary structures strongly suggesting that the α-carbon backbone tertiary structure is not affected by the introduced amino acid substitutions.

IgE-Binding Properties of Bet v 1 Triple-Patch Mutant (SEQ ID NO: 37)

The IgE-binding properties of Bet v 1 Triple-patch mutant (SEQ ID NO: 37) was compared with recombinant Bet v 1 (SEQ ID NO: 37) in a fluid-phase IgE-inhibition assay using the pool of serum IgE derived from birch allergic patients described above.

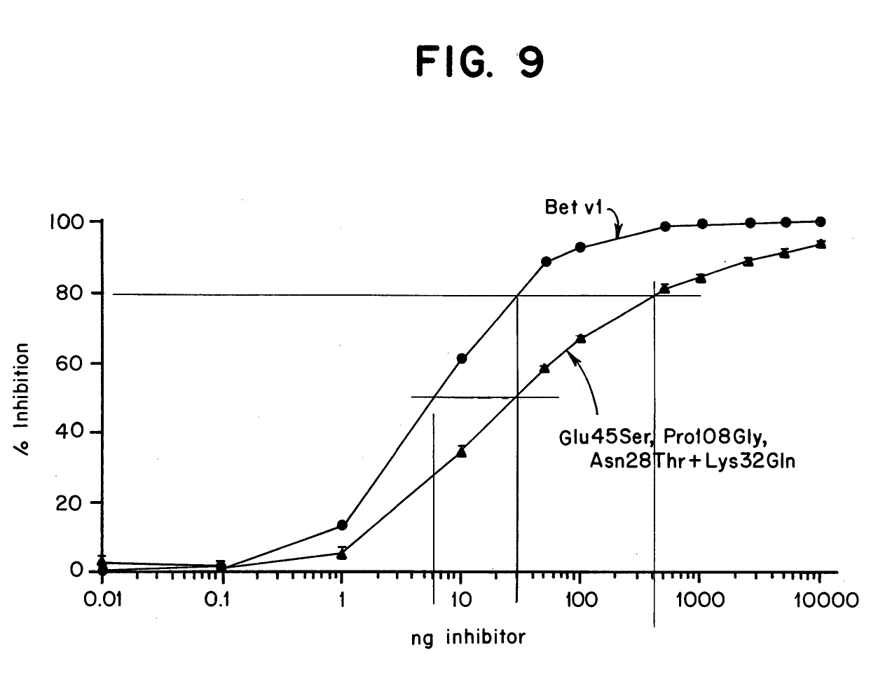

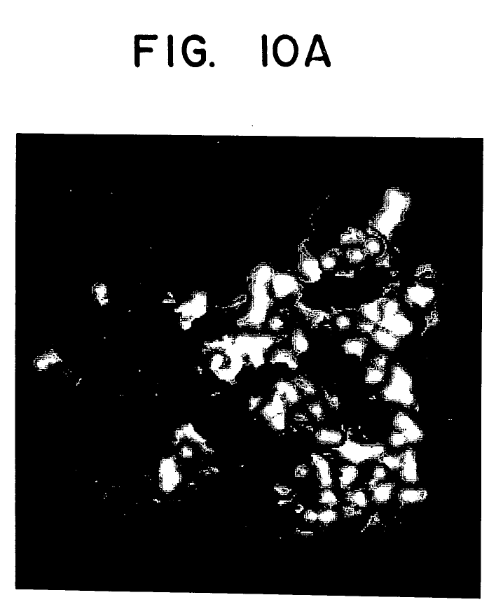

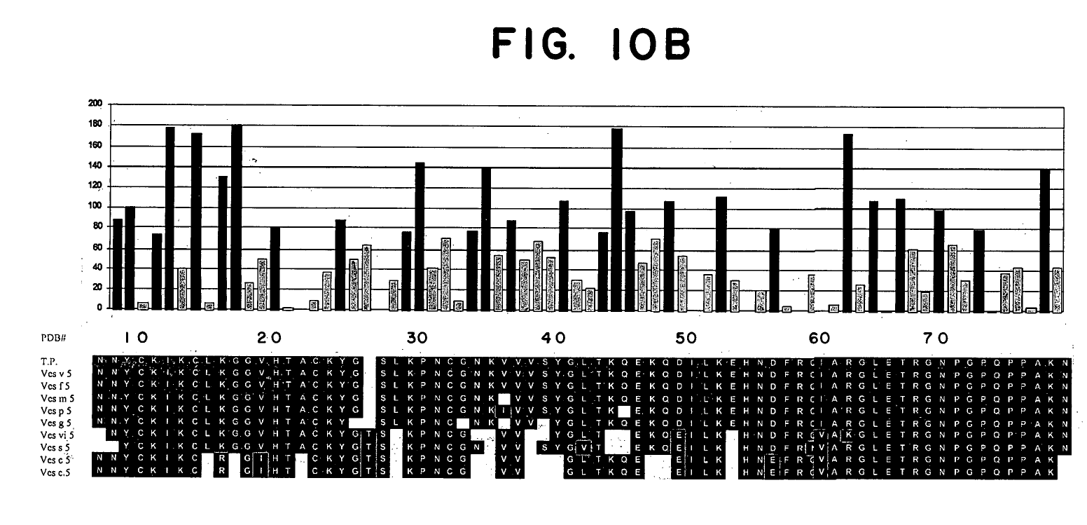

FIG. 9 shows the inhibition of the binding of biotinylated recombinant Bet v 1 (SEQ ID NO: 37) to serum IgE from a pool of allergic patients by non-biotinylated Bet v 1 (SEQ ID NO: 37) and by Bet v 1 Triple-patch mutant (SEQ ID NO: 37). In contrast to the single mutants described above, the inhibition curve of the Triple-patch mutant (SEQ ID NO: 37) is no longer parallel relative to recombinant. This shows that the substitutions introduced in the Triple-patch mutant (SEQ ID NO: 37) has changed the IgE-binding properties and epitope profile compared to recombinant. The lack of parallellity makes it difficult to quantify the decrease of the Triple-patch mutant (SEQ ID NO: 37) affinity for specific serum IgE.

Recombinant Bet v 1 (SEQ ID NO: 37) reaches 50% inhibition at about 6 ng whereas the corresponding concentration for Bet v 1 Triple-patch mutant (SEQ ID NO: 37) is 30 ng, i.e a decrease in affinity by a factor 5. However, in order to reach 80% inhibition the corresponding values are 20 ng and 400 ng, respectively, i.e a decrease by a factor 20.

T Cell Proliferation Assay Using Recombinant Bet v 1 Triple-Patch Mutant (SEQ ID NO: 37)

The analysis was carried out as described in ref. 15. It was found that recombinant Bet v 1 Triple-patch mutant (SEQ ID NO: 37) was able to induce proliferation in T cell lines from three different birch pollen allergic patients with stimulation indices similar to recombinant and naturally occurring. This suggests that the Triple-patch mutant (SEQ ID NO: 37) can initiate the cellular immune response necessary for antibody production.

### Example 2

Identification of Common Epitopes within Vespula vulgaris Venom Major Allergen Antigen 5 (SEQ ID NOS: 38-39)

Antigen 5 is one of the three vespid venom proteins, which are known allergens in man. The vespids include hornets, yellow-jacket and wasps. The other two known allergens of vespid venoms are phospholipase A₁ and hyaluronidase. Antigen 5 from Vespula vulgaris (Ves v 5) (SEQ ID NO: 39) has been cloned and expressed as recombinant protein in the yeast system (Monsalve et al. 1999, ref. 22). The three-dimensional crystal structure of recombinant Ves v 5 (SEQ ID NO: 39) has recently been determined at 1.8 Å resolution (in preparation). The main features of the structure consist of four β-strands and four α-helices arranged in three stacked layers giving rise to a “α-β-α sandwich”. The sequence identity between Antigen 5 homologous allergens from different Vespula species is about 90% suggesting presence of conserved molecular surface areas and B cell epitopes.

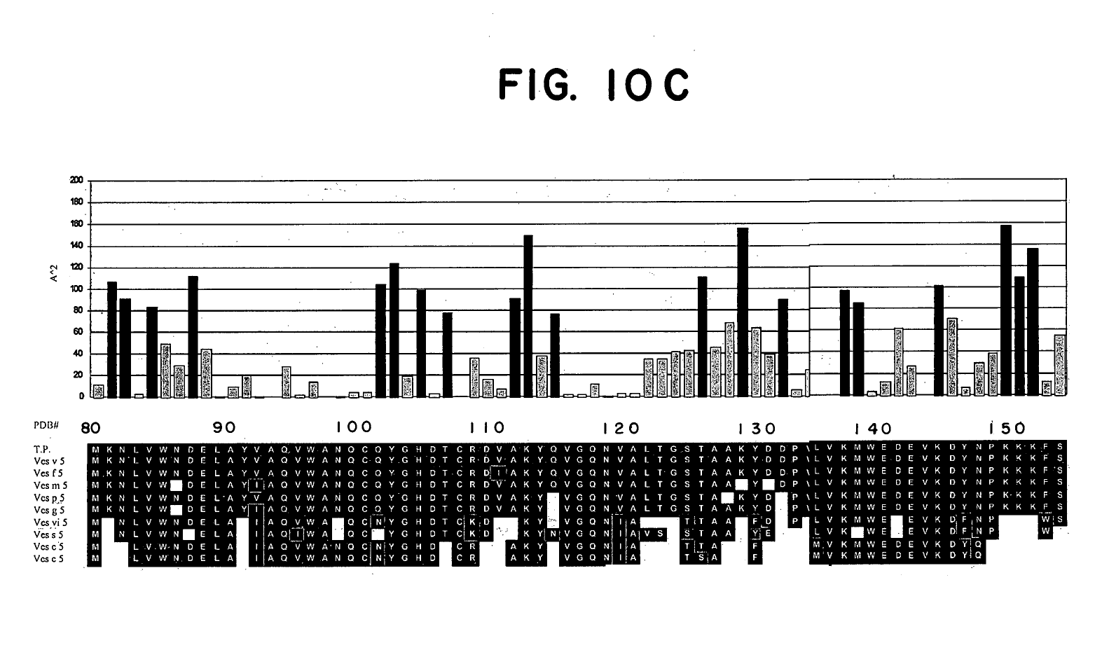

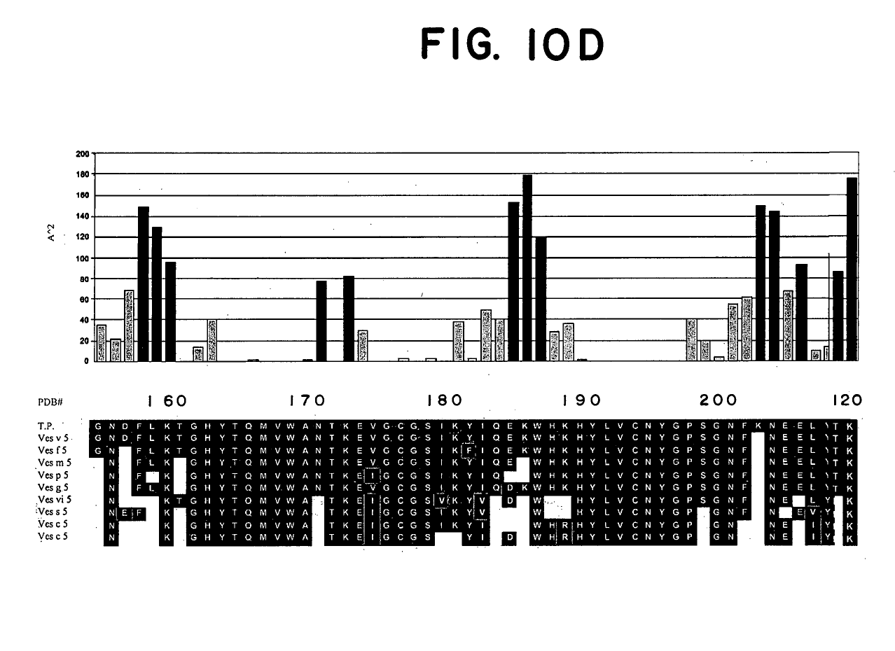

The presence and identification of common patches was performed after alignment of all known amino acid sequences, as previously described for tree pollen allergens, of the Vespula antigen 5 allergens in combination with an analysis of the molecular surface of Antigen 5 revealed by the three-dimensional structure of Ves v 5 (SEQ ID NO: 39). FIG. 10 shows solvent accessibility of individually aligned antigen 5 residues and alignment of Vespula antigen 5 sequences (left panel). On the right panel of FIG. 10 is shown the molecular surface of antigen 5 (SEQ ID NO: 39) with conserved areas among Vespula antigen 5:s coloured.

Selection of Amino Acid Residues for Site-Directed Mutagenesis

Amino acid residues for site-directed mutagenesis were selected among residues present the patches common for Vespula since modifications of these is expected to affect the binding of serum IgE from the majority of patients showing clinical Vespula allergic cross-reactivity.

The relative orientation and percentage of solvent-exposure of each amino acid residue within respective patch was calculated based on their atomic coordinates. Residues having a low degree of solvent exposure were not regarded suitable for mutagenesis due to the possible disruption of the structure or lack of antibody interaction. The remaining residues were ranked according to their degree of solvent-exposure.

Cloning of the Gene Encoding Ves v 5 (SEQ ID NO: 38)

Total RNA was isolated from venom acid glands of Vespula vulgaris vespids as described in (Fang et al. 1988, ref. 23).

First-strand cDNA synthesis, PCR amplification and cloning of the Ves v 5 (SEQ ID NO: 38) gene was performed as described in (Lu et al. 1993, ref. 24)

Subcloning into pPICZαA

The gene encoding Ves v 5 (SEQ ID NO: 38) was subsequently sub-cloned into the pPICZαA vector (Invitrogen) for secreted expression of Ves v 5 in Pichia pastoris (SEQ ID NO: 39). The gene was amplified by PCR and sub-cloned in frame with the coding sequence for the α-factor secretion signal of Saccharomyces cerevisiae. In this construct the α-factor is cleaved off, in vivo, by the Pichia pastoris Kex2 protease system during secretion of the protein.

In brief PCR was performed using Ves v 5 (SEQ ID NO: 38) as template and primers corresponding to the amino- and carboxyterminus of the protein (SEQ ID NO: 39), respectively. The primers were extended in the 5′-end to accommodate restriction sites for cloning, EcoRI and XbaI, respectively. Nucleotides encoding the Kex2 cleavage site was in this construct positioned 18 nucleotides upstream to the amino terminus of the protein (SEQ ID NO: 39), resulting in the expression of Ves v 5 with six additional amino acids, Glu-Ala-Glu-Ala-Glu-Phe, at the amino terminus (SEQ ID NO: 39).

Insertion of pPICZαA-Ves v 5 into P. pastoris

The pPICZαA vectors with the Ves v 5 gene (SEQ ID NO: 38) inserted was linearised by Sac I restriction and inserted into the AOX1 locus on the Pichia pastoris genome. Insertion was performed by homologous recombination on Pichia pastoris KM71 cells following the recommendations of Invitrogen.

In Vitro Mutagenesis

In vitro mutagenesis was performed by PCR using recombinant pPICZαA with Ves v 5 inserted as template. Each mutant Ves v 5 gene (SEQ ID NO: 38) was generated by 3 PCR reactions using 4 primers.

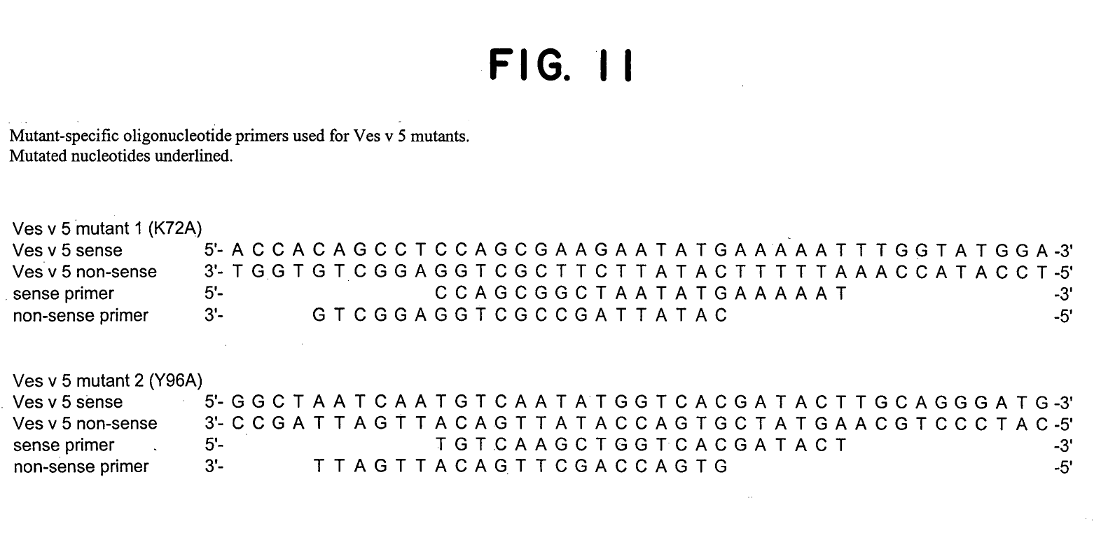

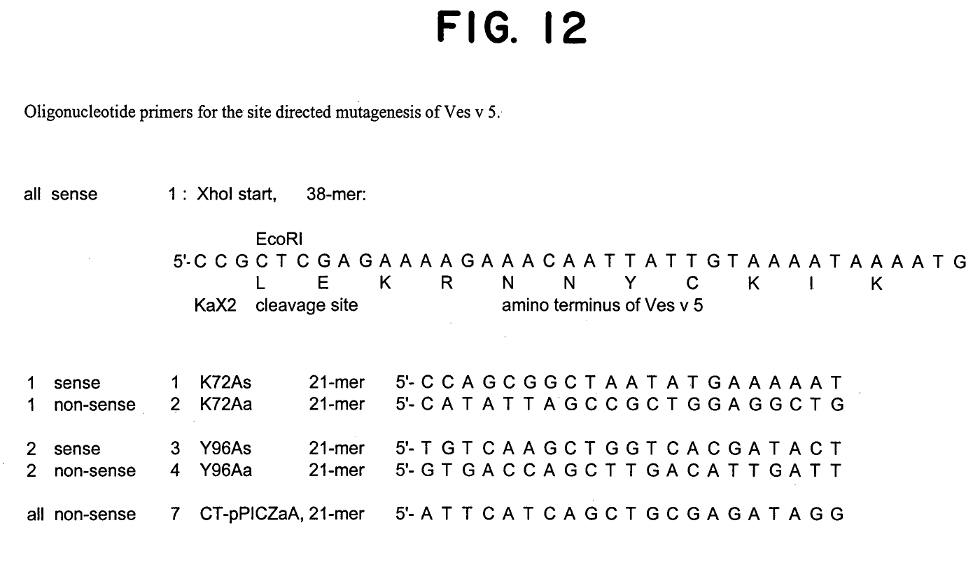

Two mutation-specific oligonucleotide primers were synthesised accommodating each mutation, one for each DNA strand, see FIGS. 11 and 12 (SEQ ID NOS: 23-35 and 40). Using the mutated nucleotide(s) as starting point both primers were extended 6-7 nucleotides in the 5′-end and 12-13 nucleotides in the 3′-end. The extending nucleotides were identical in sequence to the Ves v 5 gene (SEQ ID NO: 38) in the actual region.

Two generally applicable primers (denoted “all sense” (SEQ ID NO: 40) and “all non-sense” (SEQ ID NO: 35) in FIG. 12) were furthermore synthesised and used for all mutants. To insure expression of Ves v 5 mutants with authentic amino terminus, one primer corresponding to the amino terminus of the protein was extended in the 5′-end with a Xho I site. Upon insertion of the Ves v 5 mutant genes (SEQ ID NO: 38) into the pPICZαA vector, the Kex2 protease cleavage site was regenerated directly upstream to the amino terminus of Ves v 5 (SEQ ID NO: 39). The second primer was corresponding in sequence to a region of the pPICZαA vector positioned approximately 300 bp downstream from the Ves v 5 gene (SEQ ID NO: 38). The sequence of the primer corresponding to the amino terminus of Ves v 5 (SEQ ID NO: 39) is derived from the sense strand and the sequence of the downstream primer is derived from the non-sense strand, see FIG. 11 (SEQ ID NOS: 23-30).

Two independent PCR reactions were performed essentially according to standard procedures (Saiki et al 1988) with the exception that only 20 temperature cycles were performed in order to reduce the frequency of PCR artefacts. Each PCR reaction used pPICZαA with Ves v 5 (SEQ ID NO: 38) inserted as template and one mutation-specific and one generally applicable primer in meaningful combinations.

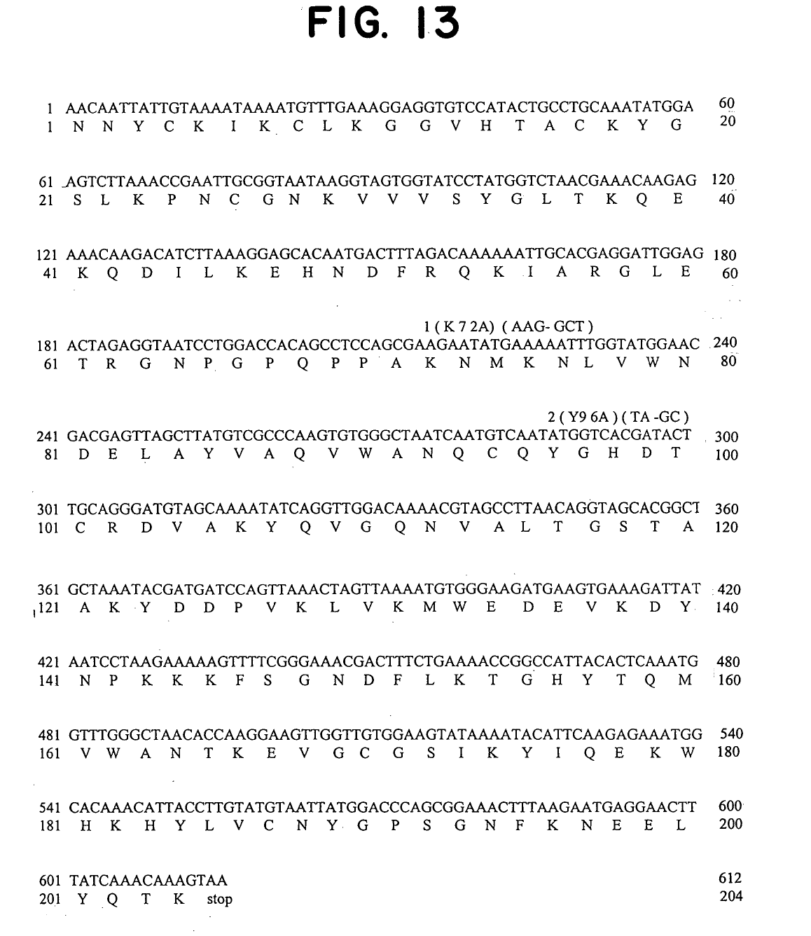

The PCR products were purified by using “Concert, Rapid PCR Purification System” (Life Technologies). A third PCR reaction was performed using the combined PCR products from the first two PCR reactions as template and both generally applicable primers. Again, 20 cycles of standard PCR were used. The PCR product was purified with the “Concert, Rapid PCR Purification System” (Life Technologies), cut with restriction enzymes (XhoI/XbaI), and ligated directionally into pPICZαA vector restricted with the same enzymes. FIG. 13 shows an overview of all Ves v 5 mutations (SEQ ID NOS: 38-39).

Insertion of pPICZαA-Ves v 5 Mutants into P. pastoris

The pPICZαA vectors with the Ves v 5 mutant genes (SEQ ID NO: 38) inserted were linearised by Sac I restriction and inserted into the AOX1 locus on the Pichia pastoris genome. Insertions were performed by homologous recombination on Pichia pastoris KM71 cells following the recommendations of Invitrogen.

Nucleotide Sequencing

Determination of the nucleotide sequence of the Ves v 5 encoding gene (SEQ ID NO: 38) was performed before and after subcloning, and following in vitro mutagenesis, respectively.

Plasmid DNA's from 10 ml of bacterial culture grown to saturation overnight in LB medium supplemented with 0.1 g/l ampicillin were purified on Qiagen-tip 20 columns and sequenced using the Sequenase version 2.0 DNA sequencing kit (USB) following the recommendations of the suppliers.

Expression and Purification of Recombinant Ves v 5 (SEQ ID NO: 39)

Recombinant yeast cells of Pichia pastoris strain KM71 were grown in 500 ml bottles containing 100 ml of pH 6.0 phosphate buffer containing yeast nitrogen base, biotin, glycerol and histidine at 30° C. with orbital shaking at 225 rpm until A₆₀₀ nm of 4-6. Cells were collected by centrifugation and re-suspended in 10 ml of similar buffered medium containing methanol in place of glycerol. Incubation was continued at 30° C. for 7 days with daily addition of 0.05 ml methanol.

Cells were harvested by centrifugation and the collected culture fluid was concentrated by ultrafiltration. After dialysis against 50 mM ammonium acetate buffer, pH 4.6, the sample was applied to a FPLC (Pharmacia) SE-53 cation exchange column equilibrated in the same buffer. The column was eluated with a 0-1.0 M NaCl, 50 mM ammonium acetate linear gradient. The recombinant Ves v 5 peak eluting at about 0.4 M NaCl was collected and dialysed against 0.02 N acetic acid. After concentration to about 10 mg/ml, the purified Ves v 5 (SEQ ID NO: 39) was stored at 4° C.

Crystallisation of Recombinant Ves v 5 (SEQ ID NO: 39)

Crystals of Ves v 5 (SEQ ID NO: 39) was grown by the vapour diffusion technique at 25° C. For crystallisation, 5 μl of 5 mg/ml Ves v 5 was mixed with 5 μl of 18% PEG 6000, 0.1 M sodium citrate, pH 6.0 and equilibrated against 1 ml of 18% PEG 6000, 0.1 M sodium citrate, pH 6.0.

X-ray diffraction data was collected at 100K from native Ves v 5 crystals (SEQ ID NO: 39) and after incorporation of heavy-atom derivatives and used to solve the three-dimensional structure of Ves v 5 (SEQ ID NO: 39), see FIG. 10 (manuscript in preparation).

Immunoelectrophoresis Using Rabbit Polyclonal Antibodies

The two Ves v 5 mutants were produced as recombinant Ves v 5 proteins (SEQ ID NO: 39) and tested for their reactivity towards polyclonal rabbit antibodies raised against recombinant Ves v 5. When analysed by rocket immunoelectrophoresis under native conditions, the rabbit antibodies were able to precipitate recombinant Ves v 5 (SEQ ID NO: 39) as well as both mutants, indicating that the mutants have conserved α-carbon backbone tertiary structure.

Inhibition of Specific serum IgE

The IgE-binding properties of Ves v 5 mutants (SEQ ID NO: 39) were compared to recombinant Ves v 5 (SEQ ID NO: 39) in a fluid-phase IgE-inhibition assay using a pool of serum IgE derived from vespid venom allergic patients.

The inhibition assay was performed as described above using biotinylated recombinant Ves v 5 (SEQ ID NO: 39) instead of Bet v 1 (SEQ ID NO: 37).

Ves v 5 Lys72Ala Mutant

Lysine in position 72 show a high degree of solvent-exposure (70%) and is located in a molecular surface patch common for Vespula antigen 5. The relative orientation and high degree of solvent exposure argued for that lysine 72 can be replaced by an alanine residue without distortion of the α-carbon backbone tertiary structure. In addition, as none of the naturally occurring isoallergen sequences have alanine in position 72, the substitution of lysine with alanine gives rise to a non-naturally occurring Ves v 5 molecule.

IgE-Binding Properties of Ves v 5 Lys72Ala Mutant (SEQ ID NO: 39)

The IgE-binding properties of Ves v 5 Lys72Ala mutant (SEQ ID NO: 39) was compared with recombinant Ves v 5 in a fluid-phase IgE-inhibition assay using the pool of serum IgE derived from birch allergic patients described above.

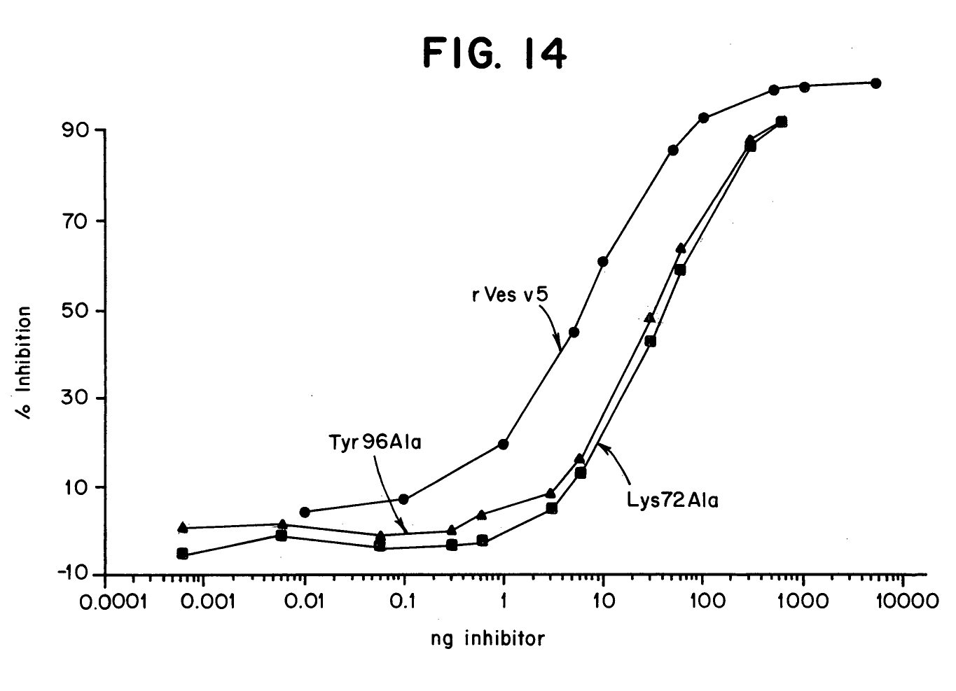

FIG. 14 shows the inhibition of the binding of biotinylated recombinant Ves v 5 (SEQ ID NO: 39) to serum IgE from a pool of allergic patients by non-biotinylated Ves v 5 (SEQ ID NO: 39) and by Ves v 5 Lys72Ala mutant (SEQ ID NO: 39).

There is a clear difference in the amount of respective recombinant proteins necessary to reach 50% inhibition of the binding to serum IgE present in the serum pool. Recombinant Ves v 5 (SEQ ID NO: 39) reaches 50% inhibition at about 6 ng whereas the corresponding concentration for Ves v 5 Lys72Ala mutant (SEQ ID NO: 39) is 40 ng. This show that the single point mutation introduced in Ves v 5 Lys72Ala mutant (SEQ ID NO: 39) lowers the affinity for specific serum IgE by a factor of about 6.

The maximum level of inhibition reached by the Ves v 5 Lys72Ala mutant (SEQ ID NO: 39) significantly lower compared to recombinant Ves v 5 (SEQ ID NO: 39). This may indicate that after the Lys72Ala substitution, some of the specific IgE present in the serum pool are unable to recognise the Ves v 5 Lys72Ala mutant (SEQ ID NO: 39).

Ves v 5 Tyr96Ala Mutant (SEQ ID NO: 39)

Tyrosine in position 96 show a high degree of solvent-exposure (65%) and is located in a molecular surface patch common for Vespula antigen 5 (SEQ ID NO: 39). The relative orientation an high degree of solvent exposure argued for that tyrosine 96 can be replaced by an alanine residue without distortion of the three-dimensional structure. In addition, as none of the naturally occurring isoallergen sequences have alanine in position 96, the substitution of tyrosine with alanine gives rise to a non-naturally occurring Ves v 5 molecule (SEQ ID NO: 39).

IgE-Binding Properties of Ves v 5 Tyr96Ala Mutant (SEQ ID NO: 39)

The IgE-binding properties of Ves v 5 Tyr96Ala mutant (SEQ ID NO: 39) was compared with recombinant Ves v 5 in a fluid-phase IgE-inhibition assay using the pool of serum IgE derived from birch allergic patients described above.

FIG. 14 shows the inhibition of the binding of biotinylated recombinant Ves v 5 (SEQ ID NO: 39) to serum IgE from a pool of allergic patients by non-biotinylated Ves v 5 (SEQ ID NO: 39) and by Ves v 5 Tyr96Ala mutant (SEQ ID NO: 39).

There is a clear difference in the amount of respective recombinant proteins necessary to reach 50% inhibition of the binding to serum IgE present in the serum pool. Recombinant Ves v 5 (SEQ ID NO: 39) reaches 50% inhibition at about 6 ng whereas the corresponding concentration for Ves v 5 Tyr96Ala mutant (SEQ ID NO: 39) is 40 ng.

This show that the single point mutation introduced in Ves v 5 Tyr96Ala mutant (SEQ ID NO: 39) lowers the affinity for specific serum IgE by a factor of about 6.

The maximum level of inhibition reached by the Ves v 5 Tyr96Ala mutant (SEQ ID NO: 39) significantly lower compared to recombinant Ves v 5 (SEQ ID NO: 39). This may indicate that after the Tyr96Ala substitution, some of the specific IgE present in the serum pool are unable to recognise the Ves v 5 Tyr96Ala mutant (SEQ ID NO: 39).

### REFERENCES

1. WO 97/30150 (Pangenetics B. V., Molecules for the induction of immunological tolerance)

2. WO 92/02621 (Biomay Biotechnik Produktions- und Handelsgesellschaft mbH, Allergens of Alder pollen and applications thereof)

3. WO 90/11293 (Immunologic Pharmaceutical Corporation, The University of North Carolina at Chapel Hill, Allergenic proteins from ragweed and uses thereof)

4. Takai T, Yokota T, Yasue M, Nishiyama C, Yuuki T, Mori A, Okudaira H, Okumura Y: “Engineering of the major house dust mite allergen Der f 2 for allergen-specific immunotherapy”. Nat Biotechnol 15, 754-758 (1997).

5. Smith A M, Chapman M D: “Localization of antigenic sites on Der p 2 using oligonucleotide-directed mutagenesis targeted to predicted surface residues”. Clin Exp Allergy 27, 593-599 (1997).

6. Aki T, Ono K, Hidaka Y, Shimonishi Y. Jyo T, Wada T, Yamashita M, Shigeta S, Murooka Y, Oka S: “Structure of IgE epitopes on a new 39-kD allergen molecule from the house dust mite, Dermatophagoides farinae”. Int Arch Allergy Immunol 103, 357-364 (1994).

7. Förster E, Dudler T, Gmachl M, Aberer W, Urbanek R, Suter M: “Natural and recombinant enzymatically active or inactive bee venom phospholipase A2 has the same potency to release histamine from basophils in patients with Hymenoptera allergy”. J Allergy Clin Immunol 95, 1229-1235 (1995).

8. Burks A W, Shin D, Cockrell G, Stanley J S, Helm R M, Bannon G A: “Mapping and mutational analysis of the IgE-binding epitopes on Ara h 1, a legume vicilin protein and a major allergen in peanut hypersensitivity”. Eur J Biochem 245, 334-339 (1997).

9. Stanley J S, King N, Burks A W, Huang S K, Sampson H, Cockrell G, Helm R M, West C M, Bannon G A: “Identification and mutational analysis of the immunodominant IgE binding epitopes of the major peanut allergen Ara h 2”. Arch Biochem Biophys 342, 244-253 (1997).

10. Ferreira F, Rohlfs A, Hoffmann-Sommergruber K, Schenk S, Ebner C, Briza P, Jilek A, Kraft D, Breitenbach M, Scheiner O: “Modulation of IgE-binding properties of tree pollen allergens by site-directed mutagenesis”. Adv Exp Med Biol 409, 127-135 (1996).

11. Ferreira F, Ebner C, Kramer B, Casari G, Briza P, Kungl A J, Grimm R, Jah-Schmid B, Breiteneder H, Kraft D, Breitenbach M, Rheinberger H-J, Scheiner O, “Modulation of IgE reactivity of allergens by site-directed mutagenesis: Potential use of hypeallergenic variants for immunotherapy”, FASEB Journal for Experimental Biology Vol. 12, No. 2, February 1998, 231-242 (1998).

12. Wiedemann P, Giehl K, Almo S C, Fedorov A A, Girvin M, Steinberger P, Rüdiger M, Ortner M, Sippl M, Dolecek C, Kraft D, Jockusch B, Valenta R: “Molecular and structural analysis of a continuous birch profilin epitope defined by a monoclonal antibody”. J Biol Chem 271, 29915-29921 (1996).

13. Alvarez A M, Fukuhara E, Nakase M, Adachi T, Aoki N, Nakamura R, Matsuda T: “Four rice seed cDNA clones belonging to the alpha-amylase/trypsin inhibitor gene family encode potential rice allergens”. Biosci Biotechnol Biochem 59, 1304-1308 (1995).

14. Colombo P, Kennedy D, Ramsdale T, Costa MA, Djro G, Izzo V, Salvadori S, Guerrini R, Cocchiara R, Mirisola M G, Wood S, Geraci D, Journal of Immunology Vol. 160, No. 6, 15 Mar. 1998, 2780-2875.

15. Spangfort M D, Ipsen H, Sparholt S H, Aasmul-Olsen S, Larsen M R, Mørtz E, Roepstorff P, Larsen J N: “Characterization of Purified Recombinant Bet v 1 with Authentic N-terminus, Cloned in Fusion with Maltose-Binding Protein”. Prot Exp Purification 8, 365-373 (1996a).

16. Ipsen H, Wihl J-Å, Petersen B N, Løwenstein H: “Specificity mapping of patients IgE response towards the tree pollen major allergens Aln g I, Bet v I and Cor a I.” Clin. Exp. Allergy 22, 391-9, (1992)

17. Gajhede M, Osmark P, Poulsen F M, Ipsen H, Larsen J N, Joost van Neerven R J, Schou C, Løwnstein H, and Spangfort M D: “X-ray and NMR structure of Bet v 1, the origin of birch pollen allergy”. Nature structural biology 3, 1040-1045 (1996).

18. Altschul S F, Gish W, Miller W, Myers E W, and Lipman D J: “Basic local alignment search tool”. J. Mol. Biol. 215, 403-410 (1990).

19. Higgins D, Thompson J, Gibson T, Thompson J D, Higgins D G, and Gibson T J: “CLUSTAL W: improving the sensitivity of progressive multiple sequence alignment through sequence weighting, position-specific gap penalties and weight matrix choice”. Nucleic Acids Res. 22, 4673-4680 (1994).

20. Saiki R K, Gelfand D H, Stoffel S, Scharf S J, Higuchi R, Horn G T, Mullis K B, Erlich H A: “Primer-directed enzymatic amplification of DNA with a thermostable DNA polymerase”. Science 239, 487-491 (1988).

21. Spangfort M D, Larsen J N, Gajhede M: “Crystallization and Preliminary X-ray Investigation at 2.0 Å Resolution of Bet v 1, a Birch Pollen Protein Causing IgE-Mediated Allergy”. PROTEINS, Struc Func Genet 26, 358-360 (1996b).

22. Monsalve R I, Lu G, and King T P: “Recombinant venom allergen, antigen 5 of yellowjacket (Vespula vulgaris) and paper wasp (Polistes annularis) by expression in bacteria or yeast” (1999) Submitted.

23. Fang K S F, Vitale M, Fehlner P and King T P: “cDNA cloning and primary structure of a white-face hornet venom allergen, antigen 5”. Proc. Natl. Acad. Sci. USA 85, 895 (1988).

24. Lu G, Villalba M, Coscia M R, Hoffman D R and King T P: “Sequence Analysis and Antigenic Cross-reactivity of a Venom Allergen, Antigen 5, from Hornets, Wasps, and Yellow Jackets”. Journal of Immunology 150, 2823-2830 (1993).

1. A method of preparing a recombinant allergen derived from a naturally occurring allergen, wherein specific IgE binding to the recombinant allergen is reduced compared to the IgE binding to said naturally occurring allergen, which method comprises:
a) identifying amino acid residues in said naturally occurring allergen which are conserved with more than 70% identity in all of the known homologous proteins within the taxonomic order from which said naturally occurring allergen originates;
b) defining at least one patch of said conserved amino acid residues which patch (i) is connected over at least 400 Å² of the surface of the three-dimensional structure of said naturally occurring allergen molecule; and (ii) comprises at least one B cell epitope, and wherein (iii) said conserved amino acid residues have a solvent accessibility of at least 20%; and
c) substituting at least one of said identified amino acid residues in said defined at least one patch with a non-conservative amino acid residue,
wherein the α-carbon backbone tertiary structure of the recombinant allergen is essentially preserved as compared with the α-carbon backbone tertiary structure of said naturally occurring allergen.

2. The method of claim 1, wherein the amino acid residues identified in step (a) are 90% or more conserved.

3. The method of claim 1, which method further comprises:
b′) ranking said conserved amino acid residues within said at least one patch in order of percent solvent accessibility; and
b″) selecting said at least one amino acid residue for substitution in step (c) from the amino acid residues having a solvent accessibility of 20-80%.

4. The method of claim 1, wherein the percent solvent accessibility is in a range from 30-80%.

5. The method of claim 1 in which the average root mean square of the atomic coordinates of the α-carbon backbone tertiary structure of the recombinant allergen deviates from the average root mean square of the atomic coordinates of the α-carbon backbone tertiary structure of said naturally occurring allergen by less than 2 Å.

6. The method of claim 1, wherein the amino acid residue substitution in step (c) is performed using site-directed mutagenesis.

7. The method of claim 1, wherein said at least one amino acid residue being substituted is a polar amino acid and said non-conservative amino acid is a polar amino acid.

8. The method of claim 1, wherein said at least one amino acid residue being substituted is a non-polar amino acid and said non-conservative amino acid is a non-polar amino acid.

9. The method of claim 1, wherein said naturally occurring allergen is an inhalation allergen or venom allergen.

10. The method of claim 1, wherein said naturally occurring allergen is a tree pollen allergen, grass pollen allergen, weed pollen allergen, herb pollen allergen, fungal allergen, dust mite allergen, animal allergen or insect allergen.

11. The method of claim 10 wherein
(a) said tree pollen allergen is from a tree taxonomically belonging to Fagales, Oleales, or Pineales;
(b) said grass pollen allergen is from a grass taxonomically belonging to Poales;
(c) said herb pollen allergen is from an herb taxonomically belonging to Aasterales;
(d) said fungal allergen is from a fungus taxonomically belonging to Alternaria or Cladosporium;
(e) said dust mite allergen is from a dust mite taxonomically belonging to Dermatophagoides;
(f) said animal allergen is from an animal taxonomically belonging to Felis, Canis, or Equus;
(g) said insect allergen is from an insect taxonomically belonging to Blatella, Periplaneta, or Hymenoptera;
(h) said weed pollen allergen is from a weed taxonomically belonging to Urticales.

12. The method of claim 11 wherein
(a) said tree taxonomically belonging to Fagales belongs taxonomically to Betula, Alnus, Corylus, or Carpinus;
(b) said tree taxonomically belonging to Oleales belongs taxonomically to Olea;
(c) said tree taxonomically belonging to Pineales belongs taxonomically to Cryptomeria;
(d) said grass taxonomically belonging to Poales belongs taxonomically to Cynodon, Dactylis, Festuca, Holcus, Lolium, Phleum, Poa, Secale or Sorghum;
(e) said herb taxonomically belonging to Aasterales belongs taxonomically to Ambrosia, Artemicia, or Urticales;
(f) said fungus taxonomically belonging to Alternaria belongs taxonomically to Alternata;
(g) said fungus taxonomically belonging to Cladosporium belongs taxonomically to herbarium;
(h) said dust mite belonging taxonomically belonging to Dermatophagoides belongs taxonomically to Farinae or Pteronyssinus;
(i) said animal taxonomically belonging to Felis belongs taxonomically to Domesticus;
(j) said animal taxonomically belonging to Canis belongs taxonomically to familiaris;
(k) said animal taxonomically belonging to Equus belongs taxonomically to caballus;
(l) said insect taxonomically belonging to Blatella belongs taxonomically to Germanica;
(m) said insect taxonomically belonging to Periplaneta belongs taxonomically to americana;
(n) said insect taxonomically belonging to Hymenoptera belongs taxonomically to Vespidae, Apidae; and
(o) said weed taxonomically belonging to Urticales belongs taxonomically to Parietaria.

13. A recombinant allergen derived from a naturally occurring allergen, wherein at least one solvent-accessible amino acid which is essentially conserved with more than 70% identity in all of the known homologous proteins within the taxonomic order from which said naturally occurring allergen originates, is substituted with an amino acid residue that is not conserved in homologous naturally-occurring allergens within said taxonomic order, wherein the α-carbon backbone tertiary structure of the recombinant allergen is conserved as compared with said naturally occurring allergen, and wherein specific IgE binding to the mutant allergen is reduced compared to IgE binding to said naturally-occurring allergen.

14. The recombinant allergen of claim 13, wherein said at least one solvent-accessible amino acid residue has a solvent accessibility of 20-80%.

15. The recombinant allergen of claim 13, in which between 1 and 5 solvent-accessible amino acid residues per at least 400 Å², are substituted.

16. The recombinant allergen of claim 13, wherein the average root mean square deviation of the atomic coordinates comparing the α-carbon backbone tertiary structures of said recombinant and said naturally occurring allergen is less than 2 Å.

17. The recombinant allergen of claim 13, wherein the specific IgE binding to said recombinant allergen is reduced by at least 5-10%.

18. The recombinant allergen of claim 17, wherein the specific IgE binding to said recombinant allergen is reduced by at least 10%.

19. The recombinant allergen of claim 13, wherein said naturally occurring allergen is an inhalation allergen or venom allergen.

20. The recombinant allergen of claim 13, wherein said naturally occurring allergen is a tree pollen allergen, grass pollen allergen, weed pollen allergen, herb pollen allergen, fungal allergen, dust mite allergen, animal allergen or insect allergen.

21. The recombinant allergen of claim 20 wherein
(a) said tree pollen allergen is from a tree taxonomically belonging to Fagales, Oleales, or Pineales;
(b) said grass pollen allergen is from a grass taxonomically belonging to Poales;
(c) said herb pollen allergen is from an herb taxonomically belonging to Aasterales;
(d) said fungal allergen is from a fungus taxonomically belonging to Alternaria or Cladosporium;
(e) said dust mite allergen is from a dust mite taxonomically belonging to Dermatophagoides;
(f) said animal allergen is from an animal taxonomically belonging to Felis, Canis, or Equus;
(g) said insect allergen is from an insect taxonomically belonging to Blatella, Periplaneta, or Hymenoptera;
(h) said weed pollen allergen is from a weed taxonomically belonging to Urticales.

22. The recombinant allergen of claim 21 wherein
(a) said tree taxonomically belonging to Fagales belongs taxonomically to Betula, Alnus, Corylus, or Carpinus;
(b) said tree taxonomically belonging to Oleales belongs taxonomically to Olea;
(c) said tree taxonomically belonging to Pineales belongs taxonomically to Cryptomeria;
(d) said grass taxonomically belonging to Poales belongs taxonomically to Cynodon, Dactylis, Festuca, Holcus, Lolium, Phleum, Poa, Secale or Sorghum;
(e) said herb taxonomically belonging to Aasterales belongs taxonomically to Ambrosia, Artemicia, or Urticales;
(f) said fungus taxonomically belonging to Alternaria belongs taxonomically to Alternata;
(g) said fungus taxonomically belonging to Cladosporium belongs taxonomically to herbarium;
(h) said dust mite belonging taxonomically belonging to Dermatophagoides belongs taxonomically to Farinae or Pteronyssinus;
(i) said animal taxonomically belonging to Felis belongs taxonomically to Domesticus;
(j) said animal taxonomically belonging to Canis belongs taxonomically to familiaris;
(k) said animal taxonomically belonging to Equus belongs taxonomically to caballus;
(l) said insect taxonomically belonging to Blatella belongs taxonomically to Germanica;
(m) said insect taxonomically belonging to Periplaneta belongs taxonomically to americana;
(n) said insect taxonomically belonging to Hymenoptera belongs taxonomically to Vespidae, Apidae; and
(o) said weed taxonomically belonging to Urticales belongs taxonomically to Parietaria.

23. The recombinant allergen of claim 20, wherein the recombinant allergen is derived from Ves v 5.

24. The recombinant allergen of claim 23, wherein the allergen has one or more amino acid substitutions selected from the group consisting of:
i) Ala at position 72 of SEQ ID NO: 39; and
ii) Ala at position 96 of SEQ ID NO: 39.

25. The recombinant allergen of claim 20, wherein the recombinant allergen is derived from Bet v 1.

26. The recombinant allergen of claim 25, wherein the allergen has one or more amino acid substitutions selected from the group consisting of:
(i) Pro at position 10 of SEQ ID NO: 37;
(ii) Gly at position 25 of SEQ ID NO: 37;
(iii) Thr at position 28 of SEQ ID NO: 37, Gln at position 32 of SEQ ID NO: 37;
(iv) Ser at position 45 of SEQ ID NO: 37;
(v) Ser at position 47 of SEQ ID NO: 37;
(vi) Asn at position 55 of SEQ ID NO: 37;
(vii) Ala at position 77 of SEQ ID NO: 37;
(viii) Gly at position 108 of SEQ ID NO: 37; and
(ix) Thr at position 28 of SEQ ID NO: 37, Gln at position 32 of SEQ ID NO: 37, Ser at position 45 of SEQ ID NO: 37, and Gly at position 108 of SEQ ID NO: 37.

27. The recombinant allergen of claim 13, which is produced by a method comprising:
a) identifying amino acid residues in said naturally occurring allergen which are conserved with more than 70% identity in all of the known homologous proteins within the taxonomic order from which said naturally occurring allergen originates;
b) defining at least one patch of said conserved amino acid residues which patch (i) is connected over at least 400 Å² of the surface of the three-dimensional structure of said naturally occurring allergen molecule; and (ii) comprises at least one B cell epitope, and wherein (iii) said conserved amino acids have a solvent accessibility of at least 20%; and
c) substituting at least one of said identified amino acid residues in said defined at least one patch with a non-conservative amino acid residue.

28. A pharmaceutical composition comprising a recombinant allergen prepared according to claim 1 and a pharmaceutically acceptable carrier.

29. A pharmaceutical composition comprising the recombinant allergen according to claim 13 and a pharmaceutically acceptable carrier.

30. A method of generating an immune response in a subject, which method comprises administering to the subject at least one recombinant allergen prepared according to claim 1 or a pharmaceutically acceptable composition comprising said at least one recombinant allergen.

31. A method of generating an immune response in a subject, which method comprises administering to the subject at least one recombinant allergen according to claim 13 or a pharmaceutically acceptable composition comprising said at least one recombinant allergen.

32. A method of vaccination or treatment of a subject, which method comprises administering to the subject at least one recombinant allergen prepared according to claim 1 or a pharmaceutically acceptable composition comprising said at least one recombinant allergen.

33. A method of vaccination or treatment of a subject, which method comprises administering to the subject at least one recombinant allergen according to claim 13 or a pharmaceutically acceptable composition comprising said at least one recombinant allergen.

34. A method of treatment, prevention or alleviation of allergic reactions in a subject, which method comprises administering to the subject at least one recombinant allergen prepared according to claim 1 or a pharmaceutically acceptable composition comprising said at least one recombinant allergen.

35. A method of treatment, prevention or alleviation of allergic reactions in a subject, which method comprises administering to the subject at least one recombinant allergen according to claim 13 or a pharmaceutically acceptable composition comprising said at least one recombinant allergen.
